(19) United States
(12) Patent Application Publication
(10) Pub. No.: US 2023/0321049 A1
(43) Pub. Date: Oct. 12, 2023

(54) COMPOSITIONS OF ENPP1 INHIBITORS AND USES THEREOF

(71) Applicant: Petragen, Inc., Davie, FL (US)

(72) Inventor: David Kolb, Davie, FL (US)

(21) Appl. No.: 18/298,059

(22) Filed: Apr. 10, 2023

Related U.S. Application Data
(60) Provisional application No. 63/328,795, filed on Apr. 8, 2022.

(51) Int. Cl. A61K 31/4184; A61K 31/517; A61K 31/352; A61K 31/472; A61K 31/18; A61K 31/4196; A61K 31/519; A61K 31/502; A61K 31/53

(52) U.S. Cl. CPC A61K 31/4184; A61K 31/517; A61K 31/352; A61K 31/472; A61K 31/18; A61K 31/4196; A61K 31/519; A61K 31/502; A61K 31/53

(57) ABSTRACT

Compositions for inhibiting ENPP1 signaling, inactivity, and/or activity are disclosed. The compositions contain a pharmaceutically acceptable carrier and a compound or a pharmaceutically acceptable salt thereof. Preferably, the compound binds to the active site of ENPP1 on the extra-cellular domain of ENPP1. Also described are methods of using the compositions. The compounds can be administered via one or more routes of administration to a subject in need thereof. The compounds are present in amounts effective to treat, prevent, or reduce one or more diseases or disorders associated with ENPP1 signaling, inactivity, and/or activity.

### CROSS-REFERENCE TO RELATED APPLICATIONS

This application claims benefit of and priority to U.S. Ser. No. 63/328,795 filed Apr. 8, 2022, which is incorporated herein by reference in its entirety.

### FIELD OF THE INVENTION

This invention is generally in the field of ectonucleotide pyrophosphatase/phosphodiesterase 1 (ENPP1) inhibition, particularly compositions containing an ENPP1 inhibitor or pharmaceutically acceptable salt thereof, and a pharmaceutically acceptable carrier, for administering to a subject in need thereof.

### BACKGROUND OF THE INVENTION

ENPP1 is a type II transmembrane glycoprotein containing two identical disulfide-bonded subunits, and possesses nucleotide pyrophosphatase and phosphodiesterase enzymatic activities. ENPP1 cleaves a variety of substrates, including phosphodiester bonds of nucleotides and nucleotide sugars and pyrophosphate bonds of nucleotides and nucleotide sugars. ENPP1 may also hydrolyze nucleoside 5′ triphosphates to their corresponding monophosphates and may also hydrolyze diadenosine polyphosphates. Further, ENPP1 is widely expressed in several tissues and plays a role in cancers; and in cardiovascular, neurological, immunological, musculoskeletal, hormonal, and hematological functions in mammals (Onyedibe, et al., Molecules 2019, 24, 4192). Therefore, ENPP1 inhibitors play a role in treating diseases and/or disorders associated with tissues that express ENPP1, where the disorder involves ENPP1 activity, inactivity, or signaling.

Most assays screening for ENPP1 inhibitors are typically performed at pH 9 to accelerate the assays, given that that is the pH at which ENPP1 is most active (Carozza, et al., Cell Chemical Biology 2020, 27, 1-12). However, ENPP1 is active at physiological conditions (such as in the range of pH 7.4 to 7.5), and an effective ENPP1 inhibitor ought to be active at physiological pH or lower, such as in the acidic microenvironment of tumors (Carozza, et al., Cell Chemical Biology 2020, 27, 1-12). Accordingly, there remains a need to identify ENPP1 inhibitors that are effective in the appropriate tissue environments.

Therefore, it is an object of the invention to provide compositions containing improved ENPP1 inhibitors.

### SUMMARY OF THE INVENTION

Disclosed are compositions and methods for modulating ENPP1 activity and/or signaling. In some forms, the compositions inhibit, and/or methods involve inhibiting, ENPP1 signaling and/or activity.

The compositions contain, and methods involve, a pharmaceutically acceptable carrier and a compound or a pharmaceutically acceptable salt thereof. Preferably, the compound inhibits ENPP1's cleaving of phosphodiester bonds of nucleotides and nucleotide sugars and pyrophosphate bonds of nucleotides and nucleotide sugars. The compounds have a structure defined by any one of Formulae I-IX, as described below. In some forms, the ENPP1 inhibitor binds to the extra-cellular domain of ENPP1, containing an active site with two Zn²⁺ ions.

The compounds can be administered via one or more routes of administration. Exemplary routes of administration are topical, mucosal, transdermal, intradermal, intravenous, intramuscular, intraperitoneal, oral, intraocular, intranasal, intracranial, or a combination thereof.

### DETAILED DESCRIPTION OF THE INVENTION

### I. Definitions

“Pharmaceutically acceptable salt” refers to the modification of the original compound by making the acid or base salts thereof. Examples of pharmaceutically acceptable salts include, but are not limited to, mineral or organic acid salts of basic residues such as amines and alkali or organic salts of acidic residues such as carboxylic acids. For original compounds containing a basic residue, pharmaceutically acceptable salts can be prepared by treating the compounds with an appropriate amount of a non-toxic inorganic or organic acid. Suitable inorganic acids include hydrochloric, hydrobromic, sulfuric, sulfamic, phosphoric, and nitric acids; suitable organic acids include acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic acids. For original compounds containing an acidic residue, pharmaceutically acceptable salts can be prepared by treating the compounds with an appropriate amount of a non-toxic base. Suitable non-toxic bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Generally, pharmaceutically acceptable salts can be prepared by reacting the free acid or base form of the original compounds with a stoichiometric amount of the appropriate base or acid, respectively, in water or in an organic solvent, or in a mixture thereof. Non-aqueous media like ether, ethyl acetate, ethanol, isopropanol, acetonitrile, or combinations thereof can be used. Lists of suitable pharmaceutically acceptable salts can be found in Remington's Pharmaceutical Sciences, 20th Ed., Lippincott Williams & Wilkins, Baltimore, M D, 2000, p. 704; and Handbook of Pharmaceutical Salts: Properties, Selection, and Use, Stahl and Wermuth, Eds., Wiley-VCH, Weinheim, 2002.

The terms “treatment” and “treating” refer to the medical management of a subject with the intent to cure, ameliorate, stabilize, or prevent one or more symptoms of a disease or disorder. This term includes active treatment toward the improvement of a disease or disorder. In addition, this term includes palliative treatment, that is, treatment designed for the relief of symptoms rather than the curing of the disease or disorder; preventative treatment, that is, treatment directed to minimizing or partially or completely inhibiting the development of the associated disease or disorder; and supportive treatment, that is, treatment employed to supplement another specific therapy directed toward the improvement of the associated disease or disorder. It is understood that treatment, while intended to cure, ameliorate, stabilize, or prevent a disease or disorder, need not actually result in the cure, amelioration, stabilization or prevention. The effects of treatment can be measured or assessed as described herein and as known in the art as is suitable for the disease or disorder involved. Such measurements and assessments can be made in qualitative and/or quantitative terms. Thus, for example, characteristics or features of a disease or disorder and/or symptoms of a disease or disorder can be reduced to any effect or to any amount.

“Lipinski's rule of five” is a rule of thumb for determining the bioavailability of orally administered drugs. The rule indicates that drug with good bioavailability, post-oral administration, general have no more than five hydrogen bond donors, no more than 10 hydrogen bond acceptors, a molecular weight less than 500 Da, and an octanol-water partition coefficient of no more than 5.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, C₁-C₂₀ heterocyclic, substituted C₁-C₂₀ heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), poly(lactic-co-glycolic acid), peptide, and polypeptide groups can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Except where specifically and expressly provided to the contrary, the term “substituted” refers to a structure, e.g., a chemical compound or a moiety on a larger chemical compound, regardless of how the structure was formed. The structure is not limited to a structure made by any specific method.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle,” “heterocyclic” and “heterocyclyl” are used interchangeably, and refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Alkyl,” as used herein, refers to the radical of saturated aliphatic groups, including straight-chain alkyl groups, branched-chain alkyl, cycloalkyl (alicyclic), alkyl substituted cycloalkyl groups, and cycloalkyl substituted alkyl. In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfoxide, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

“Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CON(R)₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, haloalkyl (such as —CF₃, —CH₂—CF₃, —CCl₃); —CN; —NCOCOCH₂CH₂; —NCOCOCHCH; —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl, sulfoxide and sulfonate), and silyl groups, as well as ethers, alkylthios, carbonyls (including ketones, aldehydes, carboxylates, and esters), haloalkyls, —CN and the like. Cycloalkyls can be substituted in the same manner.

The terms “alkenyl” and “alkynyl” refer to unsaturated aliphatic groups analogous in length and possible substitution to the alkyls described above, but that contain at least one double or triple bond respectively.

The term “substituted alkenyl” refers to alkenyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “substituted alkynyl” refers to alkynyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenyl” is art recognized, and refers to the aromatic moiety —C₆H₅, i.e., a benzene ring without one hydrogen atom.

The term “substituted phenyl” refers to a phenyl group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Amino” and “Amine,” as used herein, are art-recognized and refer to both substituted and unsubstituted amines, e.g., a moiety that can be represented by the general formula:

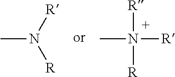

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(CH₂)ₘ—R′″. Thus, the term ‘alkylamine’ as used herein refers to an amine group, as defined above, having a substituted or unsubstituted alkyl attached thereto (i.e. at least one of R, R′, or R″ is an alkyl group).

“Carbonyl,” as used herein, is art-recognized and includes such moieties as can be represented by the general formula:

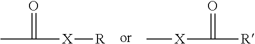

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defines as above, the moiety is also referred to as a carboxyl group. When X is oxygen and R is hydrogen, the formula represents a ‘carboxylic acid.’ Where X is oxygen and R′ is hydrogen, the formula represents a ‘formate.’ Where X is oxygen and R or R′ is not hydrogen, the formula represents an “ester”. In general, where the oxygen atom of the above formula is replaced by a sulfur atom, the formula represents a ‘thiocarbonyl’ group. Where X is sulfur and R or R′ is not hydrogen, the formula represents a ‘thioester.’ Where X is sulfur and R is hydrogen, the formula represents a ‘thiocarboxylic acid.’ Where X is sulfur and R′ is hydrogen, the formula represents a ‘thioformate.’ Where X is a bond and R is not hydrogen, the above formula represents a ‘ketone.’ Where X is a bond and R is hydrogen, the above formula represents an ‘aldehyde.’

The term “substituted carbonyl” refers to a carbonyl, as defined above, wherein one or more hydrogen atoms in R, R′ or a group to which the moiety

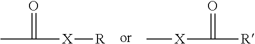

is attached, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “carboxyl” is as defined above for the formula

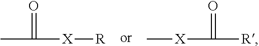

and is defined more specifically by the formula —R^{iv}COOH, wherein R^{iv} is an alkyl, alkenyl, alkynyl, cycloalkyl, heterocyclyl, alkylaryl, arylalkyl, aryl, or heteroaryl. In preferred embodiments, a straight chain or branched chain alkyl, alkenyl, and alkynyl have 30 or fewer carbon atoms in its backbone (e.g., C₁-C₃₀ for straight chain alkyl, C₃-C₃₀ for branched chain alkyl, C₂-C₃₀ for straight chain alkenyl and alkynyl, C₃-C₃₀ for branched chain alkenyl and alkynyl), preferably 20 or fewer, more preferably 15 or fewer, most preferably 10 or fewer. Likewise, preferred cycloalkyls, heterocyclyls, aryls and heteroaryls have from 3-10 carbon atoms in their ring structure, and more preferably have 5, 6 or 7 carbons in the ring structure.

The term “substituted carboxyl” refers to a carboxyl, as defined above, wherein one or more hydrogen atoms in R^{iv} are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Heteroalkyl,” as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The terms “alkoxyl” or “alkoxy,” “aroxy” or “aryloxy,” generally describe compounds represented by the formula —OR^{v}, wherein R^{v} includes, but is not limited to, substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. The term alkoxy also includes cycloalkyl, heterocyclyl, cycloalkenyl, heterocycloalkenyl, and arylalkyl having an oxygen radical attached to at least one of the carbon atoms, as valency permits.

The term “substituted alkoxy” refers to an alkoxy group having one or more substituents replacing one or more hydrogen atoms on one or more carbons of the alkoxy backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenoxy” is art recognized, and refers to a compound of the formula —OR^{v} wherein R^{v} is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Arylalkyl,” as used herein, refers to an alkyl group that is substituted with a substituted or unsubstituted aryl group. When a heteroaryl group is involved, the chemical moiety can be referred to as a “heteroarylalkyl.”

“Alkylaryl,” as used herein, refers to an aryl group that is substituted with a substituted or unsubstituted alkyl group. When a heteroaryl group is involved, the chemical moiety can be referred to as a “alkylheteroaryl.”

The terms “amide” or “amido” are used interchangeably, refer to both “unsubstituted amido” and “substituted amido” and are represented by the general formula:

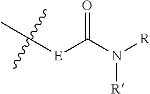

wherein, E is absent, or E is substituted or unsubstituted alkyl, substituted or unsubstituted alkenyl, substituted or unsubstituted alkynyl, substituted or unsubstituted aralkyl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ each independently represent a hydrogen atom, substituted or unsubstituted alkyl, a substituted or unsubstituted alkenyl, or —(CH₂)ₘ—R′″. When E is oxygen, a carbamate is formed. The carbamate cannot be attached to another chemical species, such as to form an oxygen-oxygen bond, or other unstable bonds, as understood by one of ordinary skill in the art.

The term “sulfonyl” is represented by the formula

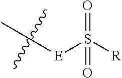

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of E and R can be substituted or unsubstituted amine, to form a “sulfonamide” or “sulfonamido.” The substituted or unsubstituted amine is as defined above.

The term “substituted sulfonyl” represents a sulfonyl in which E and R are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

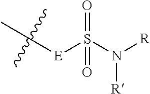

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide.

The term “sulfoxide” is represented by the formula

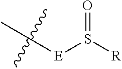

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “phosphonyl” is represented by the formula

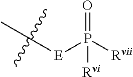

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein, independently of E, R^{vi} and R^{vii} are independently hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the P atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “substituted phosphonyl” represents a phosphonyl in which E, R^{vi} and R^{vii} are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phosphoryl” defines a phoshonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “C₃-C₂₀ cyclic” refers to a substituted or unsubstituted cycloalkyl, substituted or unsubstituted cycloalkenyl, substituted or unsubstituted cycloalkynyl that have from three to 20 carbon atoms, as geometric constraints permit. The cyclic structures are formed from single or fused ring systems. The substituted cycloalkyls, cycloalkenyls, and cycloalkynyls are substituted as defined above for the alkyls, alkenyls, and alkynyls, respectively.

The terms “hydroxyl” and “hydroxy” are used interchangeably and are represented by —OH.

The terms “thiol” and “sulfhydryl” are used interchangeably and are represented by —SH.

The term “oxo” refers to ═O.

The terms “cyano” and “nitrile” are used interchangeably to refer to —CN.

The term “nitro” refers to —NO₂.

The term “phosphate” refers to —O—PO₃.

The term “azide” or “azido” are used interchangeably to refer to —N₃.

### II. Compositions

Disclosed are compositions and methods for modulating ENPP1 activity and/or signaling. Because ENPP1 is widely expressed in several tissues and plays a role in cancers; and in cardiovascular, neurological, immunological, musculoskeletal, hormonal, and hematological functions in mammals, the disclosed compositions and methods are useful in the treatment of cancers and/or disorders associated with tissues that express ENPP1, where the disorder involves ENPP1 signaling, inactivity, and/or activity. For example, the compositions may inhibit, and/or methods may involve inhibiting, ENPP1 signaling and/or activity. For instance, ENPP1 is the major hydrolase of cyclic guanosine monophosphate-adenosine monophosphate (cGAMP) that activates the Stimulator of Interferon Genes (STING) pathway, important in anti-cancer innate immunity. Therefore, inhibiting ENPP1 can enhance treatment of cancers.

The compositions contain, and methods involve, a pharmaceutically acceptable carrier and a compound or a pharmaceutically acceptable salt thereof. In some forms, the ENPP1 inhibitor is cell impermeable. In some forms, the ENPP1 inhibitor binds to the extra-cellular domain of ENPP1. In some forms, the ENPP1 inhibitor binds to an active site of ENPP1, containing one or more (such as two) cations (such as Zn²⁺). Preferably, the compound inhibits ENPP1 activity. The ENPP1 activity includes, but is not limited to, cleaving phosphodiester bonds of nucleotides and nucleotide sugars and pyrophosphate bonds of nucleotides and nucleotide sugars, hydrolysis of nucleoside 5′ triphosphates to their corresponding monophosphates, and hydrolysis of diadenosine polyphosphates.

In some forms, the compound has a structure:

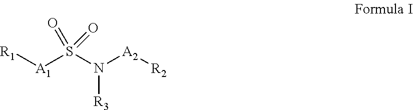

wherein:
R₁, R₂, and R₃, are independently hydrogen, substituted alkyl, unsubstituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, substituted polyheteroaralkyl, unsubstituted polyheteroaralkyl, substituted polyaralkyl, unsubstituted polyaralkyl, substituted aralkyl, unsubstituted aralkyl, or fused combinations thereof, preferably wherein the substituents include methyl, ethyl, carboxyl, acetamide, halogen, substituted urea, substituted thiazole, ═O, hydroxyl, phenyl, carboxymethyl, or a combination thereof,
A₁ and A₂ are independently absent, substituted alkyl, unsubstituted alkylene, substituted aryl, unsubstituted aryl, substituted heteroaryl, unsubstituted heteroaryl, or —NR_{A1}—,
wherein R_{A1} is hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof.
wherein (a) at least two of R₁, R₂, and R₃, are not hydrogen, and when A₁ is absent, and R₁ is unsubstituted C₁-C₃ alkyl (such as methyl) A₂ is not substituted aryl (such as substituted phenyl), (b) when A₂ is absent, and R₂ and R₃ are hydrogen, A₁ is not —NH—, (c) when R₂ and R₃ are hydrogen, and A₁ is —NH—, R₁ is not substituted 4-(ethyl)piperidine, substituted piperidine, or substituted 4-(methyl)piperidine, (d) when R₂ and R₃ are hydrogen, A₁ is —NH—, R₁ is not 4-(ethyl)piperidine, 4-(methyl)piperidine, or piperidine substituted with 6,7-dimethoxyquinazoline, 6-methoxyquinazoline, 7-methoxyquinazoline, 7-haloquinazoline, 6-haloquinazoline, 2-(2-methyl-1H-imidazol-1-yl)pyrimidin-4yl, or 6-(2-methyl-1H-imidazol-1-yl)pyrimidin-4yl, or (e) a combination of (a)-(d).

In some forms, the compounds are as described above for Formula I, except that R₁, R₂, and R₃, are independently hydrogen, substituted alkyl, unsubstituted alkyl, substituted aryl, unsubstituted aryl, substituted heteroaryl, unsubstituted heteroaryl, substituted polyaryl, unsubstituted polyaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, or fused combinations thereof.

In some forms, the compounds are as described above for Formula I, except that R₁, R₂, and R₃, are independently hydrogen, substituted alkyl, unsubstituted alkyl, substituted aryl, unsubstituted aryl, substituted heteroaryl, unsubstituted heteroaryl, unsubstituted polyaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, or fused combinations thereof.

In some forms, the compounds are as described above for Formula I, except that R₁, R₂, and R₃, are independently hydrogen, substituted alkyl, unsubstituted alkyl,

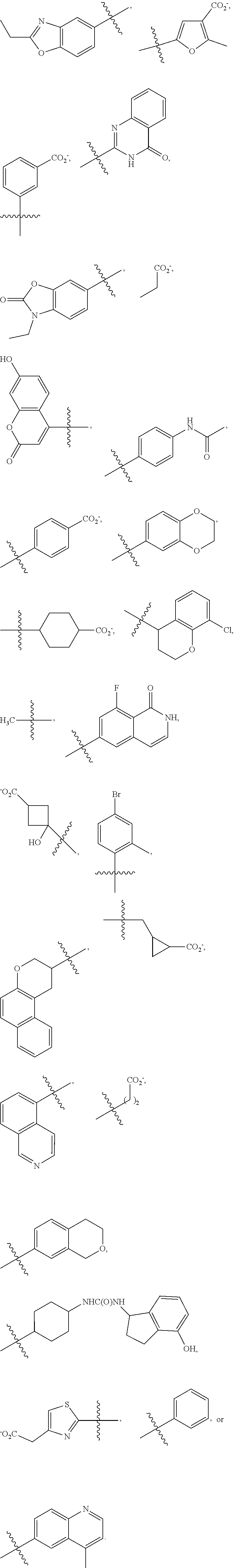

In some forms, the compounds are as described above for Formula I, except that A₁ and A₂ are independently absent, substituted alkyl, unsubstituted alkylene, substituted aryl, unsubstituted aryl, substituted heteroaryl, unsubstituted heteroaryl, or —NR_{A1}—, wherein R_{A1} is hydrogen, unsubstituted alkyl, or substituted alkyl.

In some forms, the compounds are as described above for Formula I, except that A₁ and A₂ are independently absent, substituted alkyl, unsubstituted alkylene, unsubstituted heteroaryl, or —NR_{A1}—, wherein R_{A1} is hydrogen, unsubstituted alkyl, or substituted alkyl.

In some forms, the compounds are as described above for Formula I, except that the compound has a structure:

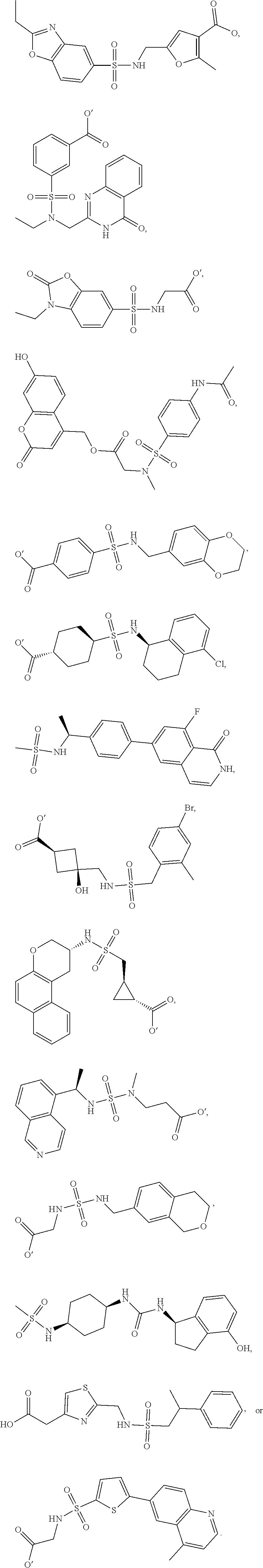

In some forms, the compound has a formula:

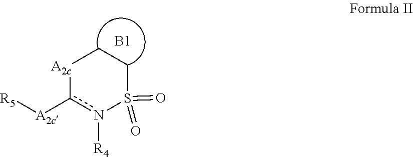

wherein:
the dashed line denotes the presence or absence of a bond,
B1 is substituted aryl, unsubstituted aryl, substituted heteroaryl, unsubstituted heteroaryl, substituted polyaryl, unsubstituted polyaryl, substituted C₃-C₂₀cycloalkyl, unsubstituted C₃-C₂₀cycloalkyl, substituted C₁-C₂₀ heterocyclyl, unsubstituted C₁-C₂₀ heterocyclyl, substituted C₃-C₂₀cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
R₄ and R₅ are independently absent, hydrogen, substituted alkyl, unsubstituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, substituted polyheteroaralkyl, unsubstituted polyheteroaralkyl, substituted polyaralkyl, unsubstituted polyaralkyl, substituted aralkyl, unsubstituted aralkyl, substituted alkyl, unsubstituted alkyl, or fused combinations thereof, preferably wherein the substituent includes a substituted amide,
A_{2c} is —NR_{A2c}— or —C(R_{A2c}R_{A2c′})—,
A_{2c′} is absent, substituted alkyl or substituted amide,
wherein R_{A2c} and R_{A2c′} are independently hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof.

In some forms, the compound is as described above for Formula II, except that the dashed line denotes the presence of a bond and R₄ is absent.

In some forms, the compound is as described above for Formula II, except that B1 is substituted aryl, unsubstituted aryl, substituted heteroaryl, unsubstituted heteroaryl, or fused combinations thereof.

In some forms, the compound is as described above for Formula II, except that B1 is unsubstituted aryl.

In some forms, the compound is as described above for Formula II, except that R₅ is substituted aryl, unsubstituted aryl, substituted heteroaryl, unsubstituted heteroaryl, or fused combinations thereof, preferably wherein the substituent includes a halogen.

In some forms, the compound is as described above for Formula II, except that R₅ is substituted aryl, unsubstituted heteroaryl, or fused combinations thereof.

In some forms, the compound is as described above for Formula II, except that R₅ has a structure:

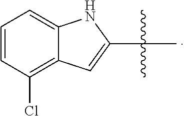 [SMILES: *c1cc2c(Cl)cccc2[nH]1]

In some forms, the compound is as described above for Formula II, except that A_{2c} is —NR_{A2c}—, wherein R_{A2c} is hydrogen.

In some forms, the compound is as described above for Formula II, except that the compound has a structure:

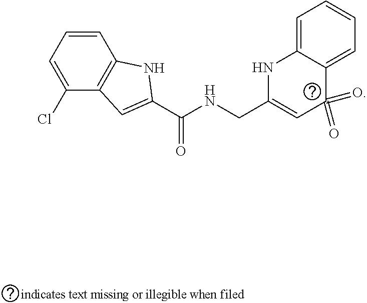

In some forms, the compound has a structure:

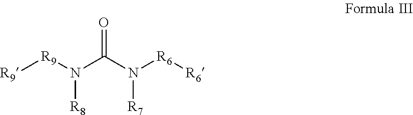

wherein:
wherein R₆ and R₉ are independently absent, substituted carbonyl, substituted alkyl, unsubstituted alkylene, substituted alkylthio, unsubstituted alkylthio, substituted alkenyl, unsubstituted alkenyl, substituted alkynyl, unsubstituted alkynyl, substituted aryl, unsubstituted aryl, substituted heteroaryl, or unsubstituted heteroaryl, preferably wherein the substituents include carboxymethyl, carboxyl, methyl, or a combination thereof,
R_{6′} and R_{9′} are independently substituted alkyl, unsubstituted alkyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, wherein at least one of R_{6′} and R_{9′} is substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, preferably wherein the substituents include methyl, ═O, tert-butyl, or a combination thereof,
wherein when R₉ is substituted alkyl or substituted alkylene, and R_{9′} is substituted aryl, the substituted aryl is not phenyl containing two or more hydroxyl groups (such as two, three, four, or five hydroxyl groups),
wherein R₇ and R₈ are independently hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof.

In some forms, the compound is as described above for Formula III, except that R₆ and R₉ are independently absent, substituted carbonyl, substituted alkyl, unsubstituted alkylene, substituted alkylthio, substituted aryl, unsubstituted aryl, substituted heteroaryl, or unsubstituted heteroaryl.

In some forms, the compound is as described above for Formula III, except that R_{6′} and R_{9′} are independently unsubstituted alkyl, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof,

In some forms, the compound is as described above for Formula III, except that at least one of R_{6′} and R_{9′} is substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula III, except that R_{6′} and R_{9′} are independently

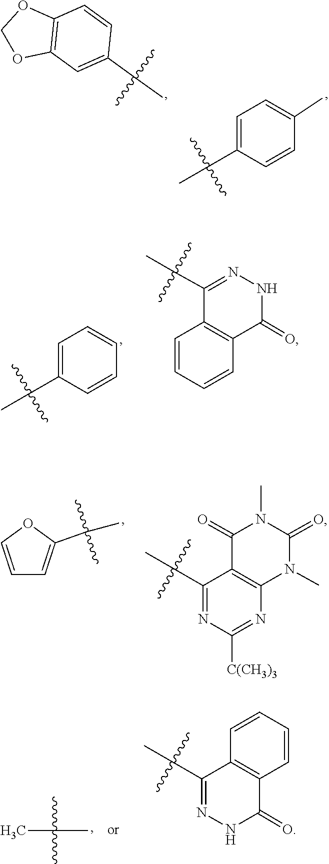

In some forms, the compound is as described above for Formula III, except that the compound has a structure:

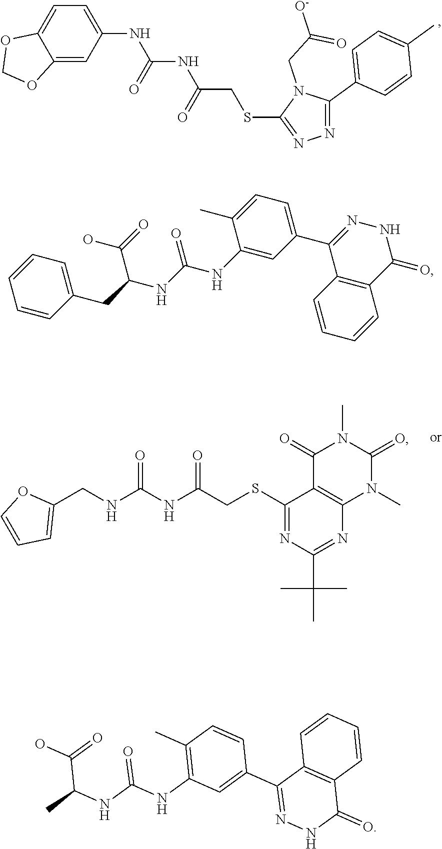 [SMILES: Cc1ccc(-c2nnc(SCC(=O)NC(=O)Nc3ccc4c(c3)OCO4)n2CC(=O)[O-])cc1 ; Cc1ccc(-c2n[nH]c(=O)c3ccccc23)cc1NC(=O)N[C@@H](Cc1ccccc1)C(=O)O ; Cn1c(=O)c2c(SCC(=O)NC(=O)NCc3ccco3)nc(C(C)(C)C)nc2n(C)c1=O ; "or" ; Cc1ccc(-c2n[nH]c(=O)c3ccccc23)cc1NC(=O)N[C@@H](C)C(=O)O]

In some forms, the compound has a structure:

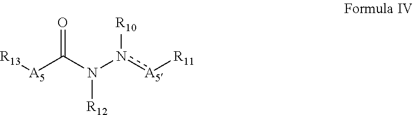

wherein:
the dashed line represents the presence of absence of a bond,
A₅ and A_{5′} are independently absent, —O—, —S—, —NR_{A5}—, substituted alkyl, unsubstituted alkyl, substituted alkylene, unsubstituted alkylene, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, preferably wherein the substituents include ═O,
wherein R_{A5}, R₁₀ and R₁₂ are independently absent, hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
R₁₁ and R₁₃ are independently substituted alkyl, unsubstituted alkyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, preferably wherein the substituents include ═O, methyl, carboxymethyloxy, or a combination thereof, wherein, (i) when -A₅-R₁₃ form a substituted alkyl, the substituted alkyl is not —CH(NH₂)CH₂OH, or (ii) when A_{5′} and R₁₀ are absent, the dashed line represents the presence of a bond, and R₁₁ is substituted aryl, the substituted aryl is not phenyl containing two or more hydroxyl groups.

In some forms, R₁₁ and R₁₃ are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, preferably wherein the substituents include ═O, methyl, carboxymethyloxy, or a combination thereof.

In some forms, the compound is as described above for Formula IV, except that A₅ and A_{5′} are independently absent, substituted alkyl, unsubstituted alkyl, substituted alkylene, unsubstituted alkylene, or substituted C₁-C₂₀ heterocyclyl.

In some forms, the compound is as described above for Formula IV, except that A₅ and A_{5′} are independently absent, substituted alkylene, or substituted C₁-C₂₀ heterocyclyl.

In some forms, the compound is as described above for Formula IV, except that R_{A5}, R₁₀ and R₁₂ are independently absent, hydrogen, unsubstituted alkyl, or substituted alkyl.

In some forms, the compound is as described above for Formula IV, except that R_{A5}, R₁₀ and R₁₂ are independently absent or hydrogen.

In some forms, the compound is as described above for Formula IV, except that R₁₁ and R₁₃ are independently substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula IV, except that R₁₁ and R₁₃ are independently substituted aryl, unsubstituted aryl, substituted C₁-C₂₀ heterocyclyl, unsubstituted C₁-C₂₀ heterocyclyl, or fused combinations thereof.

In some forms, the compound is as described above for Formula IV, except that R₁₁ and R₁₃ are independently

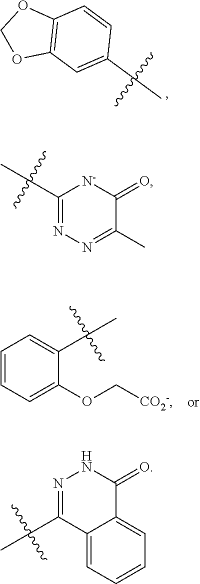

In some forms, the compound is as described above for Formula IV, except that the compound has a structure:

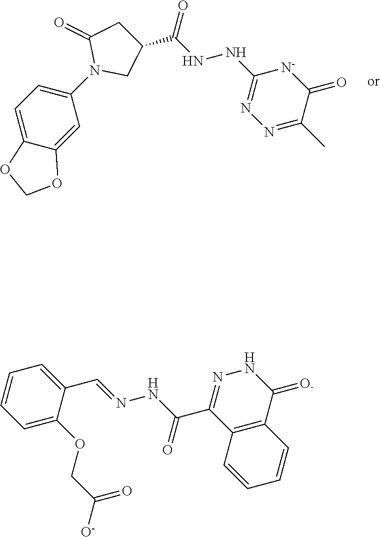 [SMILES: Cc1nnc(NNC(=O)[C@H]2CC(=O)N(c3ccc4c(c3)OCO4)C2)[n-]c1=O ; O=C([O-])COc1ccccc1/C=N/NC(=O)c1n[nH]c(=O)c2ccccc12]

In some forms, the compound has a structure:

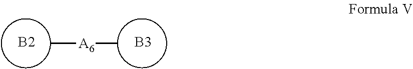

wherein:
B2 and B3 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, wherein the substituents preferably include alkoxy, ═O, carboxymethyloxy, carboxymethyl, carboxyl, methyl, halogen, phenyl, ethyne, methoxy, carbamoylmethyloxy, propyl, 3-cyclopropyl-1H-1,2,4-triazol-5-yl, iso-propyl, iso-butyl, or a combination thereof,
A₆ is a single bond, —O—, —S—, —C(O)—, —NR_{A6}— (such as —NH—), —C(O)O—, —OC(O)—, —OC(O)NR_{A6}—, —NR_{A6}C(O)O—, —OC(O)O—, —S(═O)₂—, or —S(═O)—, unsubstituted carbonyl, substituted carbonyl, unsubstituted carboxyl, substituted carboxyl, unsubstituted amino, substituted amino, unsubstituted amide, substituted amide, substituted alkyl, unsubstituted alkylthio, substituted alkylthio, unsubstituted alkyl, unsubstituted alkylene, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, wherein R_{A6} is hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl,
B2, B3, or both contain a carboxyl group, ester group, or an amide group directly or indirectly bonded thereto; a substituted alkyl (such as substituted C₁-C₆ alkyl, such as iso-propyl, iso-butyl, etc.) or unsubstituted alkyl directly bonded thereto; or a combination thereof, wherein (a) the amide group is not directly bonded to the moiety —CH(NH₂)CH₂OH or 4-halobenzyl, or (b) when the amide group is directly bonded to the moiety —CH(NH₂)CH₂OH or 4-halobenzyl, B2, B3, or both, do not contain a fused or unfused benzene group substituted with two or more hydroxyl groups (such as two, three, four, or five hydroxyl groups),
wherein (aa) A₆ is not a thioacetamide moiety (i.e., —SCH₂C(O)NH—), a thioacetamide moiety substituted with an alkyl group (i.e., —SCH(Rr)C(O)NH—, wherein Rᵣ is a unsubstituted C₁-C₂ alkyl), a sulfonyl acetamide, or a thioethan-1-amino moiety, (ab) B2 is not a purin-6-yl moiety or substituted purin-6-yl moiety, (ac) B3 is not a phenyl group or substituted phenyl group, (ad) when the substituted alkyl is indirectly bonded to B2 or B3, the substituted alkyl is not substituted with a phosphonate group, (ae) when the unsubstituted alkyl is bonded to B2 and the substituted alkyl is indirectly bonded to B2 or B3, the substituted alkyl is not substituted with a phosphonate group, (af) when A₆ contains —(CH₂)O—, B2 is not an alkoxy substituted C₆-aryl (such as methoxy substituted phenyl) or unsubstituted C₆-aryl (such as phenyl), (ag) when A₆ contains —(CH₂)O—, B3 is not a fused arylheterocyclyl (such as 2-methylisoquinolin-1(2H)-one), (ah) when A₆ contains —(CH₂)O—, B2 and B3 are not simultaneously an alkoxy substituted C₆-aryl (such as methoxy substituted phenyl) and a fused arylheterocyclyl (such as 2-methylisoquinolin-1(2H)-one), (ai) when A₆ contains —(CH₂)O—, B2 and B3 are not simultaneously unsubstituted C₆-aryl (such as phenyl) and a fused arylheterocyclyl (such as 2-methylisoquinolin-1(2H)-one) or (aj) a combination of (aa)-(ai).

In some forms, the compound is as described above for Formula V, except that B2 and B3 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, or fused combinations thereof.

In some forms, the compound is as described above for Formula V, except that B2 and B3 are independently substituted aryl, unsubstituted aryl, substituted heteroaryl, unsubstituted heteroaryl, unsubstituted polyaryl, unsubstituted polyheteroaryl, unsubstituted C₃-C₂₀ cycloalkyl, substituted C₁-C₂₀ heterocyclyl, unsubstituted C₁-C₂₀ heterocyclyl, or fused combinations thereof.

In some forms, the compound is as described above for Formula V, except that B2 and B3 are independently

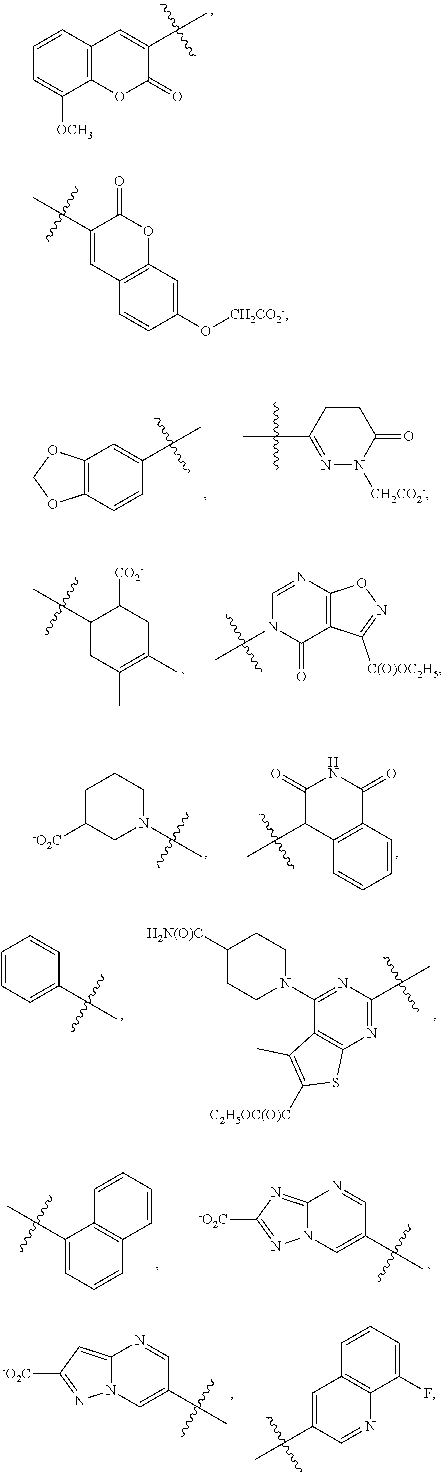

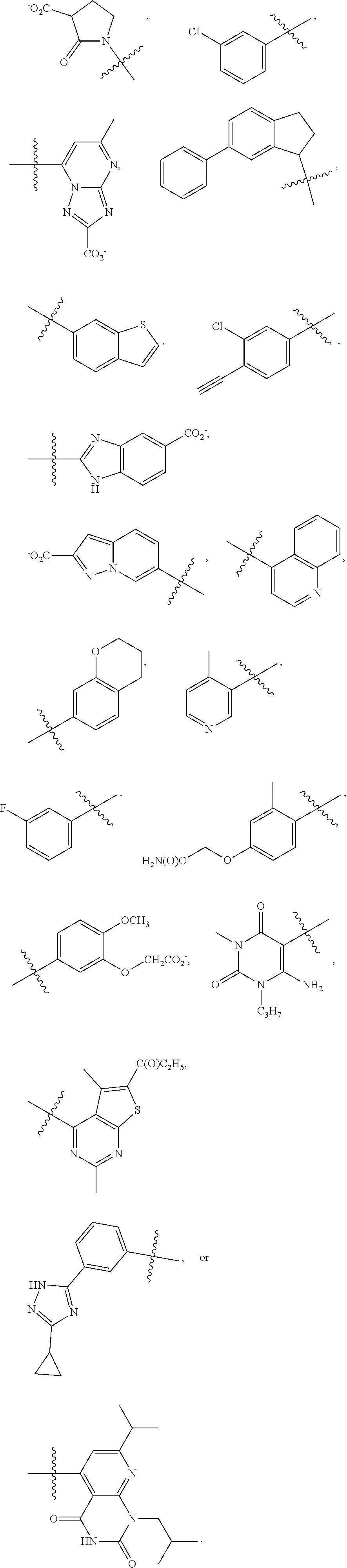

In some forms, the compound is as described above for Formula V, except that A₆ is a single bond, —NH—, unsubstituted carbonyl, substituted carbonyl, unsubstituted amide, substituted amide, substituted alkyl, unsubstituted alkylthio, substituted alkylthio, substituted aryl, unsubstituted aryl, substituted heteroaryl, unsubstituted heteroaryl, substituted C₃-C₂₀ cycloalkyl, unsubstituted C₃-C₂₀ cycloalkyl, substituted C₁-C₂₀ heterocyclyl, or unsubstituted C₁-C₂₀ heterocyclyl.

In some forms, the compound is as described above for Formula V, except that A₆ is a single bond, —NH—, substituted carbonyl, unsubstituted amide, substituted amide, substituted alkyl, unsubstituted alkylthio, substituted alkylthio, unsubstituted aryl, unsubstituted heteroaryl, substituted C₃-C₂₀ cycloalkyl, or unsubstituted C₁-C₂₀ heterocyclyl.

In some forms, the compound is as described above for Formula V, except that the compound has a structure:

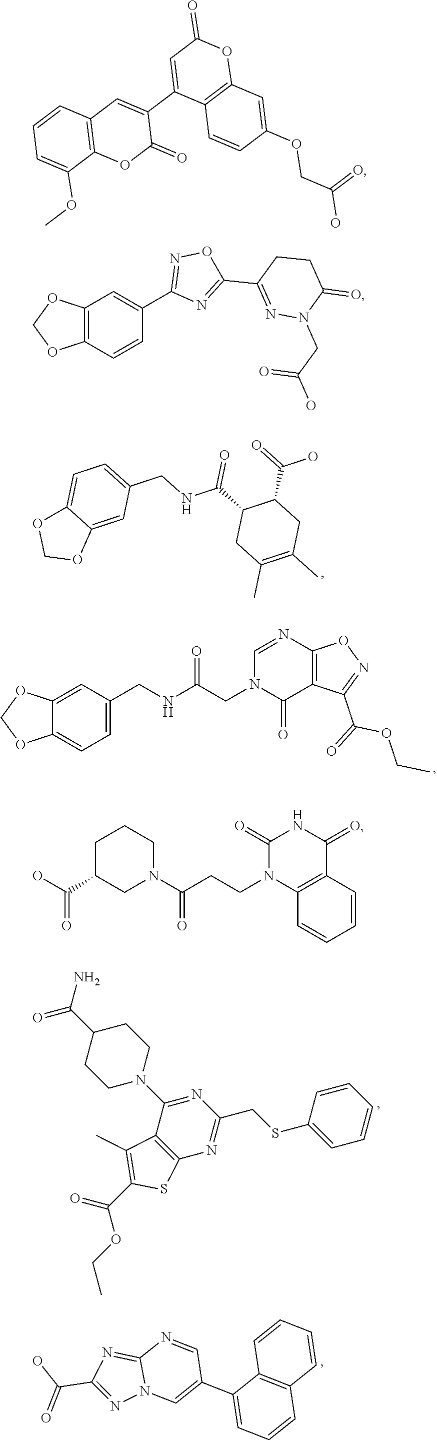

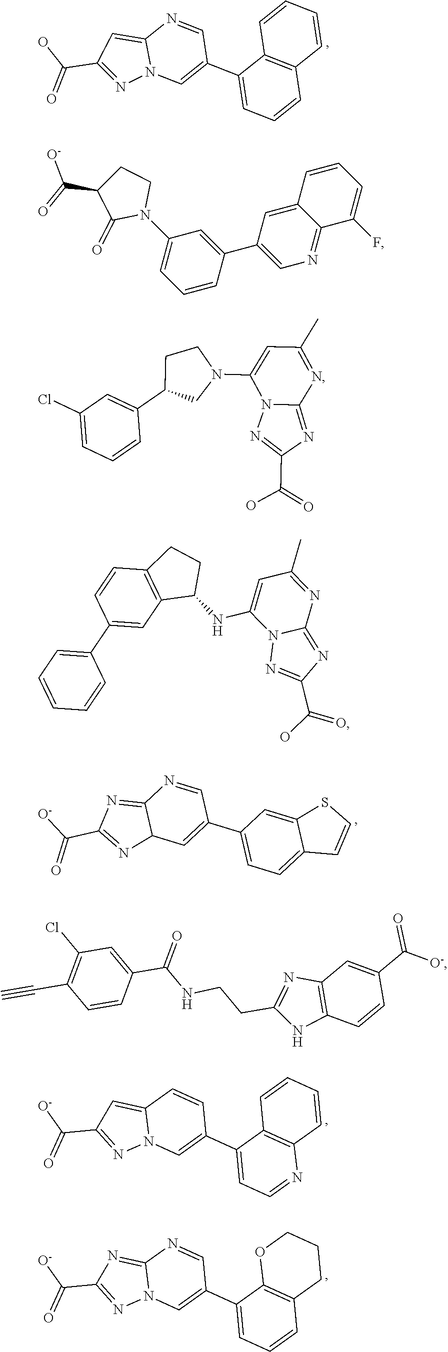

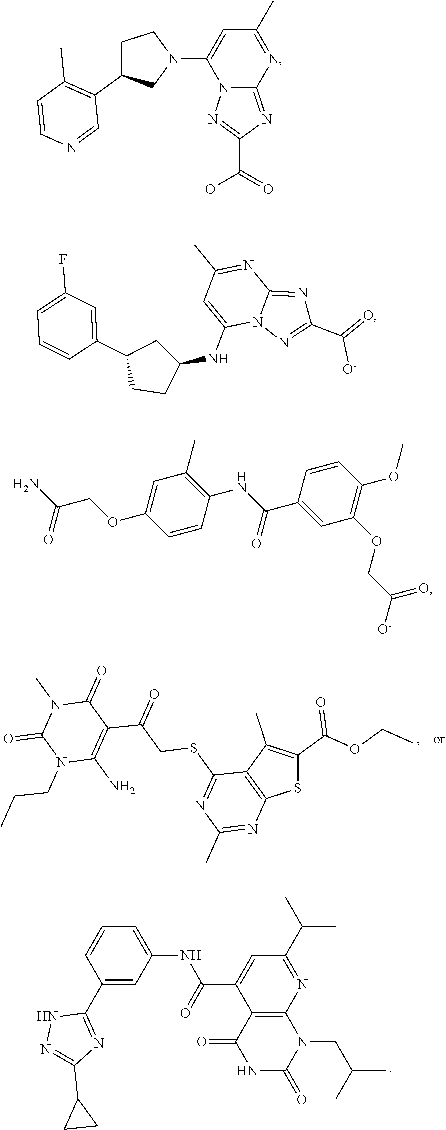 [SMILES: Cc1cc(N2CC[C@H](c3cnccc3C)C2)n2nc(C(=O)O)nc2n1 ; Cc1cc(N[C@H]2CC[C@H](c3cccc(F)c3)C2)n2nc(C(=O)[O-])nc2n1 ; COc1ccc(C(=O)Nc2ccc(OCC(N)=O)cc2C)cc1OCC(=O)[O-] ; CCCn1c(N)c(C(=O)CSc2nc(C)nc3sc(C(=O)OCC)c(C)c23)c(=O)n(C)c1=O ; CC(C)Cn1c(=O)[nH]c(=O)c2c(C(=O)Nc3cccc(-c4nc(C5CC5)n[nH]4)c3)cc(C(C)C)nc21]

In some forms, the compound has a structure:

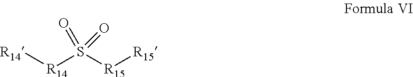

wherein:
R₁₄ and R₁₅ are organic moieties each bonded to the S(═O)₂ group via a carbon atom, and are independently unsubstituted alkyl, substituted alkyl, unsubstituted aryl, substituted aryl, substituted carbonyl, unsubstituted carbonyl, unsubstituted heteroaryl, substituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include 2-methylcyclopropan-1-yl,
R_{14′} and R_{15′} are independently absent, hydrogen, unsubstituted carboxyl, substituted carboxyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include ═O, carbamoyl, halogen, methyl, indazol-1-yl, or a combination thereof, wherein at least one of R_{14′} and R_{15′} is substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include ═O, carbamoyl, halogen, methyl, indazol-1-yl, or a combination thereof, wherein the compound is not dofetilide,
or R₁₄, R₁₅, R_{14′}, and R_{15′} combine to form a cyclic system B4:

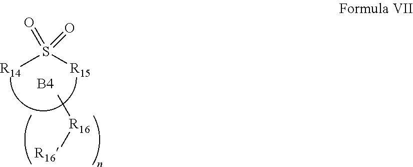

wherein:
B4 contains between four and 10 carbon atoms, between four and nine carbon atoms, or between four and eight carbon atoms, such as a thiaspiro[3.5]nonane and a tetrahydrothiophene, the carbon atoms bonded none, one, or two hydrogen atoms according to valency,
n is an integer between 1 and 10, between 1 and 9, between 1 and 8, between 1 and 7, between 1 and 6, between 1 and 5, or between 2 and 5,
each R₁₆ is independently absent, substituted alkyl, unsubstituted alkyl, substituted amide, unsubstituted amide, unsubstituted carbonyl, substituted carbonyl, unsubstituted amino, substituted amino, unsubstituted alkylthio, substituted alkylthio, unsubstituted aroxy, substituted aroxy, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
each R_{16′} is independently unsubstituted carboxyl, substituted carboxyl, unsubstituted alkyl, substituted alkyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include methyl, halogen, or a combination thereof.

In some forms, the compound is as described above for Formula VI, except that R₁₄ and R₁₅ are organic moieties each bonded to the S(═O)₂ group via a carbon atom, and are independently unsubstituted alkyl, substituted alkyl, unsubstituted aryl, substituted aryl, substituted carbonyl, unsubstituted carbonyl, unsubstituted heteroaryl, substituted heteroaryl, or fused combinations thereof.

In some forms, the compound is as described above for Formula VI, except that R₁₄ and R₁₅ are organic moieties each bonded to the S(═O)₂ group via a carbon atom, and are independently unsubstituted alkyl, substituted alkyl, unsubstituted aryl, substituted aryl, substituted carbonyl, unsubstituted heteroaryl, or substituted heteroaryl.

In some forms, the compound is as described above for Formula VI, except that R_{14′} and R_{15′} are independently absent, hydrogen, unsubstituted carboxyl, substituted carboxyl, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof, wherein at least one of R_{14′} and R_{15′} is substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula VI, except that R_{14′} and R_{15′} are independently absent, hydrogen, unsubstituted carboxyl, substituted aryl, unsubstituted aryl, substituted heteroaryl, unsubstituted heteroaryl, substituted C₁-C₂₀ heterocyclyl, or fused combinations thereof, wherein at least one of R_{14′} and R_{15′} is substituted aryl, unsubstituted aryl, substituted heteroaryl, unsubstituted heteroaryl, substituted C₁-C₂₀ heterocyclyl, or fused combinations thereof.

In some forms, the compound is as described above for Formula VI, except that R_{14′} and R_{15′} are independently absent, hydrogen,

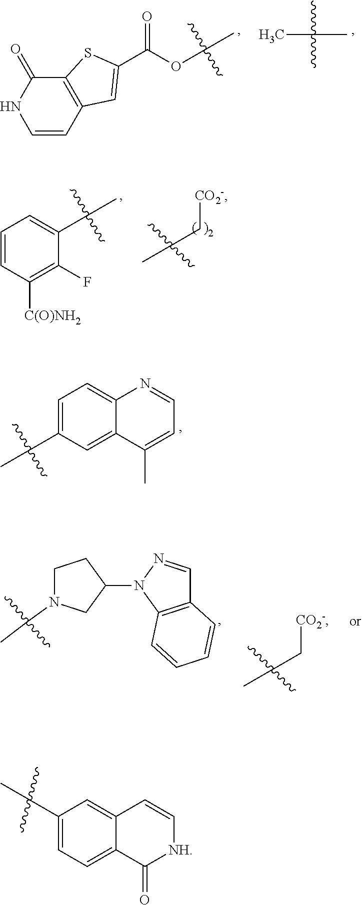

In some forms, the compound is as described above for Formula VII, except that B4 contains between four and eight carbon atoms, such as a thiaspiro[3.5]nonane and a tetrahydrothiophene.

In some forms, the compound is as described above for Formula VII, except that n is an integer between 2 and 5.

In some forms, the compound is as described above for Formula VII, except that each R₁₆ is independently absent, substituted alkyl, unsubstituted alkyl, substituted amide, or unsubstituted amide.

In some forms, the compound is as described above for Formula VII, except that each R₁₆ is independently absent, substituted alkyl, or substituted amide.

In some forms, the compound is as described above for Formula VII, except that each R_{16′} is independently unsubstituted carboxyl, substituted carboxyl, unsubstituted alkyl, substituted alkyl, substituted aryl, unsubstituted aryl, substituted heteroaryl, unsubstituted heteroaryl, or fused combinations thereof.

In some forms, the compound is as described above for Formula VII, except that each R_{16′} is independently unsubstituted carboxyl, unsubstituted alkyl, substituted alkyl, substituted aryl, unsubstituted heteroaryl, or fused combinations thereof.

In some forms, the compound is as described above for Formula VII, except that each R_{16′} is independently

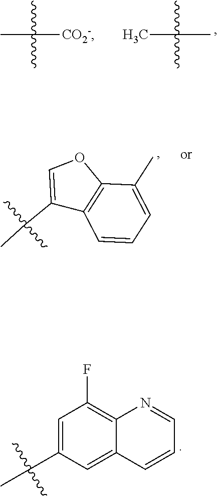

In some forms, the compound is as described above for Formula VII, except that the compound has a structure:

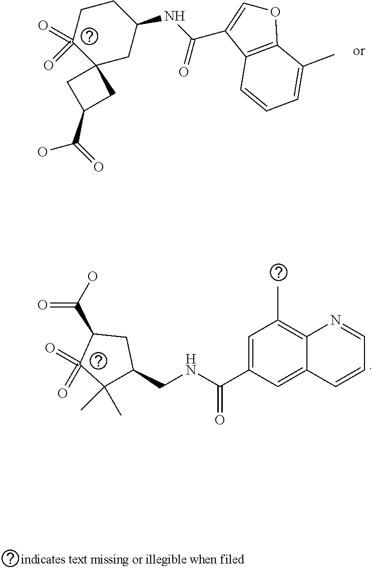

In some forms, the compound has a structure:

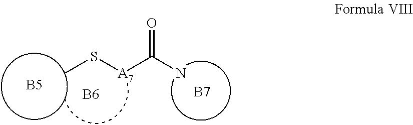

wherein:
the dashed line denotes the presence or absence of a ring system, and when present the ring is denoted B6,
A₇ is a carbon atom bonded to one or no hydrogen atom according to valency, unsubstituted alkyl, substituted alkyl, or unsubstituted alkylene, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
B5, B6 when present, and B7 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include carbamoyl, methyl, 2-hydroxyphenyl, 3-methoxybenylamine, methoxy, or a combination thereof.

In some forms, the compound is as described above for Formula VIII, except that the dashed line denotes the absence of a ring system and B6 is not present, and A₇ is unsubstituted alkyl, substituted alkyl, or unsubstituted alkylene.

In some forms, the compound is as described above for Formula VIII, except that B5 and B7 are independently substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula VIII, except that B5 and B7 are independently

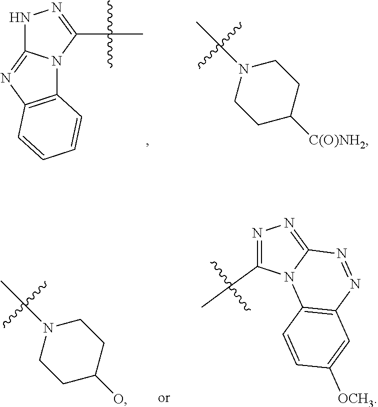

In some forms, the compound is as described above for Formula VIII, except that the compound has a structure:

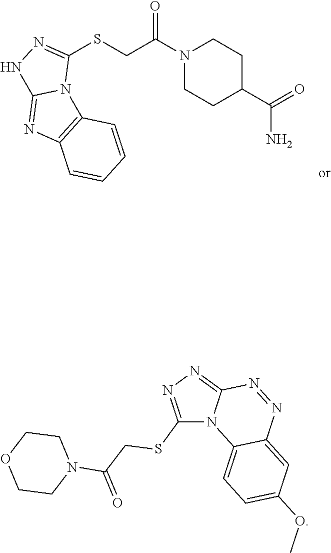 [SMILES: NC(=O)C1CCN(C(=O)CSc2n[nH]c3nc4ccccc4n23)CC1 ; COc1ccc2c(c1)nnc1nnc(SCC(=O)N3CCOCC3)n12]

In some forms, the compound is as described above for Formula VIII, except that the dashed line denotes the presence of a ring system and B6 is present, wherein:
A₇ is a carbon atom bonded to one or no hydrogen atom according to valency,
B6 is substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula VIII, except that B6 is substituted heteroaryl.

In some forms, the compound is as described above for Formula VIII, except that B5 and B7 are independently substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula VIII, except that B5 and B7 are independently substituted heteroaryl, substituted C₁-C₂₀ heterocyclyl, or fused combinations thereof.

In some forms, the compound is as described above for Formula VIII, except that B5 and B7 are independently

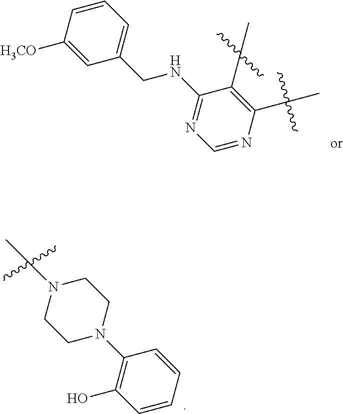 [SMILES: *c1ncnc(NCc2cccc(OC)c2)c1* ; *N1CCN(c2ccccc2O)CC1]

In some forms, the compound is as described above for Formula VIII, except that the compound has a structure:

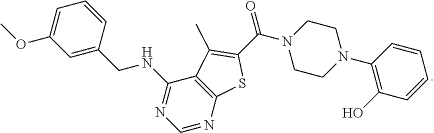 [SMILES: COc1cccc(CNc2ncnc3sc(C(=O)N4CCN(c5ccccc5O)CC4)c(C)c23)c1]

In some forms, the compound has a structure:

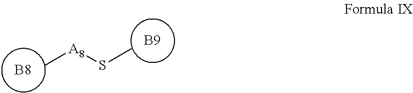

wherein:
A₈ is substituted alkylene (such as substituted C₁-C₇ alkylene), unsubstituted alkylene (such as unsubstituted C₁-C₇ alkylene), wherein -A₈-S— is not a thioacetamide (i.e., —SCH₂C(O)NH—), a thioacetamide moiety substituted with an alkyl group (i.e., —SCH(Rᵣ)C(O)NH—, wherein Rᵣ is a unsubstituted C₁-C₂ alkyl) a sulfonyl acetamide, or a thioethan-1-amino moiety,
B8 and B9 are independently unsubstituted aroxy, substituted aroxy, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, preferably wherein the substituents include halophenyl, ═O, methyl, ethyl methanoate, 4-carbamoylpiperidin-1-yl, or a combination thereof.

In some forms, the compound is as described above for Formula IX, except that B8 and B9 are independently unsubstituted aroxy, substituted aroxy, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

In some forms, the compound is as described above for Formula IX, except that B8 and B9 are independently unsubstituted aroxy, substituted aryl, substituted heteroaryl, unsubstituted heteroaryl, unsubstituted C₁-C₂₀ heterocyclyl, or fused combinations thereof.

In some forms, the compound is as described above for Formula IX, except that B8 and B9 are independently

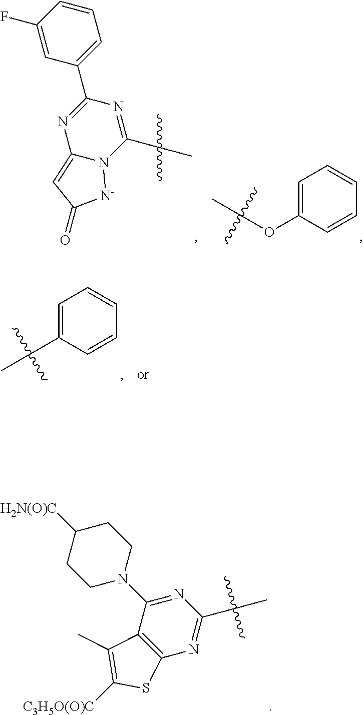

In some forms, the compound is as described above for Formula IX, except that the compound has a structure:

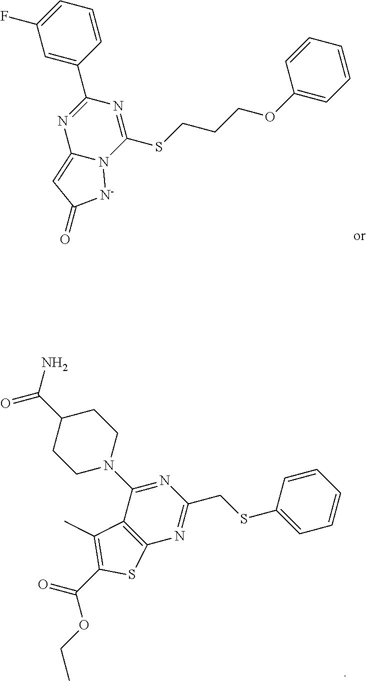 [SMILES: O=c1cc2nc(-c3cccc(F)c3)nc(SCCCOc3ccccc3)n2[n-]1 ; CCOC(=O)c1sc2nc(CSc3ccccc3)nc(N3CCC(C(N)=O)CC3)c2c1C]

Every compound within the above definition of Formulae I-IX is intended to be and should be considered to be specifically disclosed herein. Further, every subgroup that can be identified within the above definition is intended to be and should be considered to be specifically disclosed herein. As a result, it is specifically contemplated that any compound or subgroup of compounds can be either specifically included for or excluded from use or included in or excluded from a list of compounds. For example, any one or more of the compounds described herein, with a structure depicted herein, or referred to in the Tables or the Examples herein can be specifically included, excluded, or combined in any combination, in a set or subgroup of such compounds. Such specific sets, subgroups, inclusions, and exclusions can be applied to any aspect of the compositions and methods described here. For example, a set of compounds that specifically excludes one or more particular compounds can be used or applied in the context of compounds per se (for example, a list or set of compounds), compositions including the compound (including, for example, pharmaceutical compositions), any one or more of the disclosed methods, or combinations of these. Different sets and subgroups of compounds with such specific inclusions and exclusions can be used or applied in the context of compounds per se, compositions including one or more of the compounds, or any of the disclosed methods. All of these different sets and subgroups of compounds—and the different sets of compounds, compositions, and methods using or applying the compounds—are specifically and individually contemplated and should be considered as specifically and individually described. As an example, any of the species of “R” groups (such as R₁, R₂, R₃, R₄, R₅, R₆, R₇, etc), as defined above, can be specifically included or excluded, as a group or individually, from any position in the compounds per se (for example, a list or set of compounds), from compounds in compositions (including, for example, pharmaceutical compositions), or any one or more of the disclosed methods, or combinations of these. Further, specific compounds can be excluded from the list of compounds. Preferably, the compound is as disclosed above for any one of Formulae I-IX, except that the compound is not a species disclosed in U.S. Pat. No. 10,689,376 to Vankayalapati, et al.; Carozza, et al., Cell Chemical Biology 2020, 27, 1-12; Gangar, et al., Bioorg. Chem. 2022, 119, 105549; Onyedibe, et al., Molecules 2019, 24, 4192; Patel, et al., Bioorg. Med. Chem. Lett. 2009, 19, 3339-3343; WO2022/056068 by Deb, et al., U.S. Patent Application Publication 2021/0369747 by Li, et al., U.S. Pat. No. 10,624,882 to Bosanac, et al., WO2020/081923 by Bosanac, et al., or U.S. Patent Application Publication 2010/0317691 by Wong, et al. The contents of these documents are herein incorporated in their entirety, by reference.

In some forms, the compound is as described above for any of Formulae I-IX, except that the compound has a topological polar surface area (i) between 70 Å and 140 Å, or (ii) greater than 140 Å.

In some forms, the compound is as described above for any of Formulae I-IX, except that the compound has a molecular weight (i) between 200 Da and 500 Da, or (ii) greater than 500 Da and no more than 2,500 Da.

In some forms, the compound is as described above for any of Formulae I-IX, except that the compound has one or more of hydrogen bond donors, hydrogen bond acceptors, molecular weight, and octanol-water partition coefficient non-conforming with Lipinski's rule of five.

In some forms, the compound is as described above for any of Formulae I-IX, except that the compound is:
(i) in a solution;
(ii) in a suspension;
(iii) in a gel; or
(iv) encapsulated and/or bound to an implant, nanoparticle, microparticle, nanogel, microgel.

### III. Methods of Making and Reagents Therefor

The compounds in the methods and compositions described herein can be synthesized using methods known to those of skill in the art of organic chemistry synthesis. In some forms, some of the compounds can be purchased from one or more commercial vendors.

### IV. Methods of Using

As discussed in more detail elsewhere herein, ENPP1 is widely expressed in several tissues and has been implicated in cancers, as well as cardiovascular, neurological, immunological, musculoskeletal, hormonal, and hematological functions, as well as periodontal diseases and gingivitis in mammals. Therefore, the disclosed compositions and methods are suitable for use in the treatment of diseases or disorders associated with tissues that express ENPP1, where the disease or disorder involves ENPP1 activity. For example, the compositions may modulate (such as inhibit), and/or methods may involve modulating (such as inhibiting), ENPP1 activity and/or signaling.

The methods typically include administering to a subject in need thereof an effective amount of a disclosed compound, composition, or formulation. As used herein, the term “effective amount” or “therapeutically effective amount” means a dosage sufficient to treat, inhibit, or alleviate one or more symptoms of a disease state or disorder being treated or to otherwise provide a desired pharmacologic and/or physiologic effect. The precise dosage will vary according to a variety of factors such as subject-dependent variables (such as age, immune system health, etc.), the disease, disorder, and the treatment being effected.

In some forms, methods that reduce ENPP1 signaling and/or enzymatic activity are provided. For example, a method of reducing ENPP1 signaling and/or enzymatic activity can include administering a subject in need thereof an effective amount of a disclosed compound, composition, or formulation to reduce ENPP1 signaling and/or activity. In some forms, the formulation is provided in an amount effective to reduce nucleotide and/or nucleotide binding to ENPP1. In some forms, the formulation reduces activation of an ENPP1 pathway. The activity may include modulating phosphodiester bond hydrolysis, pyrophosphate bond hydrolysis, or a combination thereof. In some forms, the activity may include inhibiting cyclic guanosine monophosphate-adenosine monophosphate (cGAMP) hydrolysis, nucleoside 5′ triphosphate hydrolysis (such as ATP hydrolysis), diadenosine polyphosphate hydrolysis, or a combination thereof.

The effective amount of the compound can be ascertained from assays investigating the inhibition of ENPP1-nucleotide/nucleotide binding compared to a control that does not contain the compound, as determined by an assay that detects fluorescence polarization. In some forms, the compound has a half-maximal inhibitory concentration (IC₅₀) of inhibiting a ENPP1-nucleotide/nucleotide sugar interaction of less than 1,000 PM, or less than 100 μM, or less than 10 μM, or less than 1 μM, or less than 0.1 μM, or less than 0.01 μM or less than 0.001 μM; for example, 0.001 μM-1,000 μM, or 0.001 μM-100 μM, or 0.001 μM-10 μM, or 0.01 μM-1,000 μM, or 0.01 μM-100 μM, or 0.01 μM-10 μM, or 0.1 μM-1,000 μM, or 0.1 μM-100 μM, or 0.1 μM-10 μM, or 1 μM-1,000 μM, or 1 μM-100 μM, or 1 μM-10 μM, or any subrange or specific number therebetween.

Additional Formulations

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

These formulations can take the form of solutions, suspensions, emulsion, gel, cream, lotion, transdermal patch, oils, tablets, pills, capsules, powders, sustained-release formulations such as nanoparticles, microparticles, etc., and the like.

i. Parenteral Formulations

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

If for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

If needed, the formulation can be buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

1. Controlled Release Formulations

The parenteral formulations described herein can be formulated for controlled release including immediate release, delayed release, extended release, pulsatile release, and combinations thereof.

(a) Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethylcellulose, hydroxypropylcellulose, methylcellulose, and carboxymethylcellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

(b) Method of Making Nano- and Microparticles

Methods for preparing microparticles and nanoparticles include, but are not limited to, self-assembly; crosslinking; solvent evaporation and/or emulsion encapsulation (such as single emulsion solvent evaporation or multi-emulsion solvent evaporation); hot melt particle formation; solvent removal; spray drying; phase inversion; microfluidics; coacervation; low temperature casting; molecular dispersion or phase separated dispersion techniques; or solid phase encapsulation techniques.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

ii. Enteral Formulations

Oral formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier so as to provide the proper form to the patient based on the mode of administration to be used.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

1. Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another form, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(a) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred forms, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred forms, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred form, the acrylic polymer is an acrylic resin lacquer such as that which is commercially available from Rohm Pharma under the tradename EUDRAGIT®. In further preferred forms, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(b) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGIT® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### EXAMPLES

### Example 1: Screening for ENPP1 Inhibitors

### Materials and Methods

(i) In Silico Screening

The Schrödinger Suite software package was used to perform in silico screening. Structure-based drug design (SBDD) was performed using an ENPP1 crystal structure, having PDB ID: 6WET, 6WEU, 6WEV, 6WEW, or 6WFJ using the ENAMINE library of compounds. Ligand-based drug design (LBDD) was also performed with pharmacophore and/or shape-based screening. A subset of compounds from the SBDD and LBDD was selected and scored using free energy perturbation (such as using FEP+) calculations. A subset of the compounds was selected from the FEP+ scoring and advanced for in vitro screening.

(ii) In Vitro Screening

ENPP1 was from R&D systems, 6136-EN-10. 10 μM ATP (Part #2053, BellBrook Labs) was used as substrate. ENPP1 concentration was set to 50 μM. ENPP1 reaction buffer contained 25 mM Tris (pH 7.5), 10 mM MgCl2, 0.01% Brij-35.

Test compounds: 239 test compounds were screened. 10 mM stocks in DMSO were prepared by UW-Madison Small Molecule Screening Facility. Control compounds were Suramin (S2671) from Sigma and ENPP1-n-1 (31764) from Cayman Chemical. The Transcreener AMP²/GMP² FP Assay was used to measure [AMP] produced. Compounds were pre-incubated with ENPP1 for 30 min at room temperature to ensure enzyme-inhibitor (E*I) complex formation. ATP was added, and reactions were incubated at room temperature for 2h. Stop and Detect Solution was added, and the plates were incubated for a further 60 min to develop signal. Assays were run in Corning Assay plate 384-well low-volume black plates, and fluorescence polarization (FP) was read on a CLARIOstar Plus plate reader.

Control compounds: Compounds were dispensed into assay-ready plates prepared by UW-Madison Small Molecule Screening Facility, including control compounds (Suramin and ENPP1-n-1).

Inhibition assays were performed at three concentrations: 40 μM, 20 PM, and 10 μM i.e., a three-point screen (n=2). Based on the results of the three-point screen, a subset of compounds was selected and screened at eight concentrations, i.e., an eight-point screen (n=2). Compounds were titrated into plates at 100 μM maximum concentration, diluted serially 1:3 to create 8-point dose response reactions. Assays were set up as described above.

Z-scores were calculated from the raw FP data in Microsoft Excel. FP data were converted to [AMP] using standard curves fit in GraphPad Prism with “log(inhibitor) vs response” non-linear regression. Percent inhibition was calculated in Microsoft Excel.

### Results

The results from the eight-point screenings for the 10-μM concentration point, are shown in Table 1.

TABLE 1
In vitro screening against ENPP1

| | 10 uM | Z- | | | Ring | |
|---|---|---|---|---|---|---|
| Label | inhib | Score | IC₅₀ uM | TPSA | Count | Mol Wt |
| PTGN-33918 | b | 3.3 | +++ | 123.92 | 5 | 384.379 |
| PTGN-10419 | b | 3.9 | +++ | 125.47 | 3 | 363.371 |
| PTGN-03820 | b | 4.7 | +++ | 147.5 | 4 | 468.471 |
| PTGN-03221 | b | 5.1 | +++ | 136.85 | 4 | 371.333 |
| PTGN-48122 | b | 5.6 | +++ | 119.01 | 4 | 393.327 |
| PTGN-22742 | b | 7 | +++ | 93.3 | 3 | 363.416 |
| PTGN-22243 | b | 6.8 | +++ | 83.21 | 4 | 289.274 |
| PTGN-12123 | b | 3.3 | +++ | 70.59 | 4 | 395.439 |
| PTGN-20724 | b | 1.8 | ++ | 130.18 | 4 | 343.275 |
| PTGN-83844 | b | 5.6 | ++ | 70.32 | 4 | 288.286 |
| PTGN-44425 | b | 3.9 | ++ | 109.38 | 4 | 358.427 |
| PTGN-30326 | b | 2.4 | ++ | 90.82 | 5 | 489.601 |
| PTGN-35545 | b | 5.1 | ++ | 73.33 | 4 | 349.341 |
| PTGN-61646 | a | 4.5 | ++ | 86.45 | 4 | 356.793 |
| PTGN-74247 | a | 4.8 | ++ | 95.53 | 3 | 372.85 |
| PTGN-27448 | a | 3.5 | ++ | 95.24 | 5 | 384.419 |
| PTGN-19527 | b | 2.9 | + | 136.57 | 3 | 365.325 |
| PTGN-11549 | a | 3.7 | + | 117.36 | 2 | 350.347 |
| PTGN-47228 | b | 2.8 | + | 123.26 | 3 | 386.409 |
| PTGN-36629 | b | 3.2 | + | 94.74 | 4 | 360.399 |
| PTGN-57550 | a | 3.4 | + | 79.03 | 3 | 360.41 |
| PTGN-23067 | a | 3.9 | | 140.01 | 2 | 387.368 |
| PTGN-09266 | a | 2.9 | | 96.36 | 2 | 354.453 |
| PTGN-44363 | a | 4.3 | | 107.56 | 2 | 299.328 |
| PTGN-32353 | a | 5.4 | | 106.53 | 2 | 391.263 |
| PTGN-37261 | a | 4.4 | | 102.43 | 2 | 336.393 |
| PTGN-57133 | b | 3.3 | | 121.44 | 2 | 299.284 |
| PTGN-14052 | a | 3.8 | | 97.91 | 3 | 366.784 |
| PTGN-32254 | a | 5.1 | | 87.16 | 3 | 354.407 |
| PTGN-85440 | a | 2.4 | | 104.76 | 3 | 348.356 |
| PTGN-23139 | b | 1.8 | | 143.22 | 3 | 460.464 |
| PTGN-66058 | a | 5.1 | | 116.26 | 3 | 393.416 |
| PTGN-18437 | b | 2.6 | | 127.01 | 3 | 365.369 |
| PTGN-67036 | b | 2.9 | | 141.12 | 3 | 460.516 |
| PTGN-61535 | b | 3.3 | | 115.3 | 3 | 344.347 |
| PTGN-93732 | b | 3.4 | | 139.17 | 3 | 491.595 |
| PTGN-71430 | b | 5.3 | | 87.69 | 3 | 330.36 |
| PTGN-80264 | a | 4.2 | | 107.53 | 3 | 367.471 |
| PTGN-01068 | a | 3.9 | | 112.4 | 3 | 350.376 |
| PTGN-45869 | a | 3.9 | | 99.19 | 3 | 361.424 |
| PTGN-33355 | a | 5.6 | | 116.51 | 4 | 390.437 |
| PTGN-84557 | a | 3.6 | | 70.32 | 4 | 288.286 |
| PTGN-69760 | a | 3.3 | | 103.42 | 4 | 388.836 |
| PTGN-33762 | a | 4.3 | | 99.34 | 4 | 337.363 |
| PTGN-12765 | a | 3 | | 95.24 | 4 | 354.365 |
| PTGN-59456 | a | 5 | | 95.53 | 4 | 360.411 |
| PTGN-70559 | a | 4.7 | | 92.44 | 4 | 295.278 |
| PTGN-94870 | a | 3.9 | | 97.71 | 4 | 358.423 |
| PTGN-93538 | b | 2.6 | | 98.41 | 4 | 470.62 |
| PTGN-18434 | b | 2.4 | | 134.78 | 4 | 400.347 |
| PTGN-88031 | b | 2.5 | | 127.01 | 4 | 441.467 |
| PTGN-55851 | b | 6.8 | | 83.21 | 4 | 295.303 |
| PTGN-94641 | a | 2.3 | | 138.42 | 5 | 487.564 |

a between 1% and 30% inhibition at 10 μM
b greater than 30% inhibition at 10 μM
+++ : IC₅₀ between 1 and 20 μM
++: IC₅₀ between 21 and 40 μM
+: IC₅₀ greater than 41 μM

Those skilled in the art will recognize, or be able to ascertain using no more than routine experimentation, many equivalents to the specific embodiments of the invention described herein. Such equivalents are intended to be encompassed by the following claims.

I claim:

1. A composition comprising a pharmaceutically acceptable carrier and a compound or a pharmaceutically acceptable salt thereof, wherein the compound has a structure
(i) Formula I: 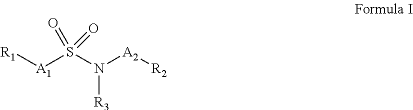
wherein:
R₁, R₂, and R₃, are independently hydrogen, substituted alkyl, unsubstituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, substituted polyheteroaralkyl, unsubstituted polyheteroaralkyl, substituted polyaralkyl, unsubstituted polyaralkyl, substituted aralkyl, unsubstituted aralkyl, or fused combinations thereof,
A₁ and A₂ are independently absent, substituted alkyl, unsubstituted alkylene, substituted aryl, unsubstituted aryl, substituted heteroaryl, unsubstituted heteroaryl, or —NR_{A1}—,
wherein R_{A1} is hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
wherein (a) at least two of R₁, R₂, and R₃, are not hydrogen, and when A₁ is absent, and R₁ is unsubstituted C₁-C₃ alkyl (such as methyl) A₂ is not substituted aryl (such as substituted phenyl), (b) when A₂ is absent, and R₂ and R₃ are hydrogen, A₁ is not —NH—, (c) when R₂ and R₃ are hydrogen, and A₁ is —NH—, R₁ is not substituted 4-(ethyl)piperidine, substituted piperidine, or substituted 4-(methyl)piperidine, (d) when R₂ and R₃ are hydrogen, A₁ is —NH—, R₁ is not 4-(ethyl)piperidine, 4-(methyl)piperidine, or piperidine substituted with 6,7-dimethoxyquinazoline, 6-methoxyquinazoline, 7-methoxyquinazoline, 7-haloquinazoline, 6-haloquinazoline, 2-(2-methyl-1H-imidazol-1-yl)pyrimidin-4yl, or 6-(2-methyl-1H-imidazol-1-yl)pyrimidin-4yl, or (e) a combination of (a)-(d),
(ii) Formula II: 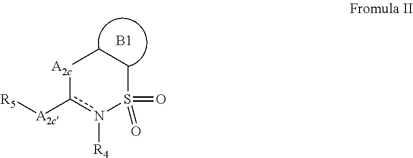
wherein:
the dashed line denotes the presence or absence of a bond,
B1 is substituted aryl, unsubstituted aryl, substituted heteroaryl, unsubstituted heteroaryl, substituted polyaryl, unsubstituted polyaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
R₄ and R₅ are independently absent, hydrogen, substituted alkyl, unsubstituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, substituted polyheteroaralkyl, unsubstituted polyheteroaralkyl, substituted polyaralkyl, unsubstituted polyaralkyl, substituted aralkyl, unsubstituted aralkyl, substituted alkyl, unsubstituted alkyl, or fused combinations thereof,
A_{2c} is —NR_{A2c}— or —C(R_{A2c}R_{A2c′})—,
A_{2c′} is absent, substituted alkyl or substituted amide,
wherein R_{A2c} and R_{A2c′} are independently hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
(iii) Formula III: 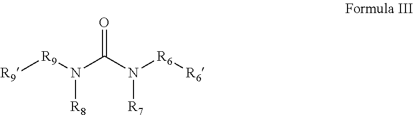
wherein:
wherein R₆ and R₉ are independently absent, substituted carbonyl, substituted alkyl, unsubstituted alkylene, substituted alkylthio, unsubstituted alkylthio, substituted alkenyl, unsubstituted alkenyl, substituted alkynyl, unsubstituted alkynyl, substituted aryl, unsubstituted aryl, substituted heteroaryl, or unsubstituted heteroaryl,
R_{6′} and R_{9′} are independently substituted alkyl, unsubstituted alkyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, wherein at least one of R_{6′} and R_{9′} is substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀cycloalkyl, unsubstituted C₃-C₂₀cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
wherein when R₉ is substituted alkyl or substituted alkylene, and R_{9′} is substituted aryl, the substituted aryl is not phenyl containing two or more hydroxyl groups (such as two, three, four, or five hydroxyl groups),
wherein R₇ and R₈ are independently hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
(iv) Formula IV: 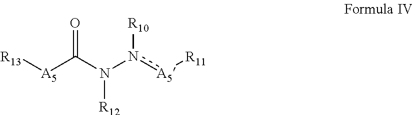
wherein:
the dashed line represents the presence of absence of a bond,
A₅ and A_{5′} are independently absent, —O—, —S—, —NR_{A5}—, substituted alkyl, unsubstituted alkyl, substituted alkylene, unsubstituted alkylene, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl,
wherein R_{A5}, R₁₀ and R₁₂ are independently absent, hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
R₁₁ and R₁₃ are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof,
(v) Formula V: 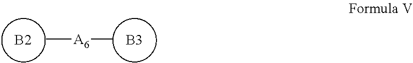
wherein:
B2 and B3 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
A₆ is a single bond, —O—, —S—, —C(O)—, —NR_{A6}— (such as —NH—), —C(O)O—, —OC(O)—, —OC(O)NR_{A6}—, —NR_{A6}C(O)O—, —OC(O)O—, —S(═O)₂—, or —S(═O)—, unsubstituted carbonyl, substituted carbonyl, unsubstituted carboxyl, substituted carboxyl, unsubstituted amino, substituted amino, unsubstituted amide, substituted amide, substituted alkyl, unsubstituted alkylthio, substituted alkylthio, unsubstituted alkyl, unsubstituted alkylene, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, wherein R_{A6} is hydrogen, unsubstituted alkyl, substituted alkyl, substituted alkenyl, unsubstituted alkenyl, substituted alkynyl, unsubstituted alkynyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl,
B2, B3, or both contain a carboxyl group, ester group, or an amide group directly or indirectly bonded thereto; a substituted alkyl (such as substituted C₁-C₆ alkyl, such as iso-propyl, iso-butyl, etc.) or unsubstituted alkyl directly bonded thereto; or a combination thereof, wherein (a) the amide group is not directly bonded to the moiety —CH(NH₂)CH₂OH or 4-halobenzyl, or (b) when the amide group is directly bonded to the moiety —CH(NH₂)CH₂OH or 4-halobenzyl, B2, B3, or both, do not contain a fused or unfused benzene group substituted with two or more hydroxyl groups (such as two, three, four, or five hydroxyl groups),
wherein (aa) A₆ is not a thioacetamide moiety (i.e., —SCH₂C(O)NH—), a thioacetamide moiety substituted with an alkyl group (i.e., —SCH(Rᵣ)C(O)NH—, wherein Rᵣ is a C₁-C₂ alkyl), a sulfonyl acetamide, or a thioethan-1-amino moiety, (ab) B2 is not a purin-6-yl moiety or substituted purin-6-yl moiety, (ac) B3 is not a phenyl group or substituted phenyl group, (ad) when the substituted alkyl is indirectly bonded to B2 or B3, the substituted alkyl is not substituted with a phosphonate group, (ae) when the unsubstituted alkyl is bonded to B2 and the substituted alkyl is indirectly bonded to B2 or B3, the substituted alkyl is not substituted with a phosphonate group, (af) when A₆ contains —(CH₂)O—, B2 is not an alkoxy substituted C₆-aryl (such as methoxy substituted phenyl) or unsubstituted C₆-aryl (such as phenyl), (ag) when A₆ contains —(CH₂)O—, B3 is not a fused arylheterocyclyl (such as 2-methylisoquinolin-1(2H)-one), (ah) when A₆ contains —(CH₂)O—, B2 and B3 are not simultaneously an alkoxy substituted C₆-aryl (such as methoxy substituted phenyl) and a fused arylheterocyclyl (such as 2-methylisoquinolin-1(2H)-one), (ai) when A₆ contains —(CH₂)O—, B2 and B3 are not simultaneously unsubstituted C₆-aryl (such as phenyl) and a fused arylheterocyclyl (such as 2-methylisoquinolin-1(2H)-one), or (aj) a combination of (aa)-(ai),
(vi) Formula VI: 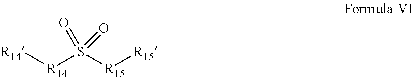
wherein:
R₁₄ and R₁₅ are organic moieties each bonded to the S(═O)₂ group via a carbon atom, and are independently unsubstituted alkyl, substituted alkyl, unsubstituted aryl, substituted aryl, substituted carbonyl, unsubstituted carbonyl, unsubstituted heteroaryl, substituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
R_{14′} and R_{15′} are independently absent, hydrogen, unsubstituted carboxyl, substituted carboxyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, wherein at least one of R_{14′} and R_{15′} is substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof, wherein the compound is not dofetilide,
or R₁₄, R₁₅, R_{14′}, and R_{15′} combine to form a cyclic system B4: 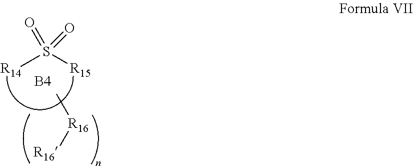
wherein:
B4 contains between four and 10 carbon atoms, between four and nine carbon atoms, or between four and eight carbon atoms, such as a thiaspiro[3.5]nonane and a tetrahydrothiophene, the carbon atoms bonded none, one, or two hydrogen atoms according to valency,
n is an integer between 1 and 10, between 1 and 9, between 1 and 8, between 1 and 7, between 1 and 6, between 1 and 5, or between 2 and 5,
each R₁₆ is independently absent, substituted alkyl, unsubstituted alkyl, substituted amide, unsubstituted amide, unsubstituted carbonyl, substituted carbonyl, unsubstituted amino, substituted amino, unsubstituted alkylthio, substituted alkylthio, unsubstituted aroxy, substituted aroxy, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
each R_{16′} is independently unsubstituted carboxyl, substituted carboxyl, unsubstituted alkyl, substituted alkyl, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
(vii) Formula VIII: 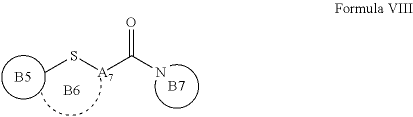
wherein:
the dashed line denotes the presence or absence of a ring system, and when present the ring is denoted B6,
A₇ is a carbon atom bonded to one or no hydrogen atom according to valency, unsubstituted alkyl, substituted alkyl, or unsubstituted alkylene, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
B5, B6 when present, and B7 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof,
or
(viii) Formula IX: 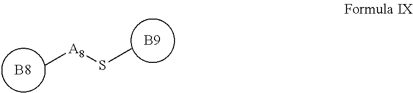
wherein:
A₈ is substituted alkylene (such as substituted C₁-C₇ alkylene), unsubstituted alkylene (such as unsubstituted C₁-C₇ alkylene), wherein -A₈-S— is not a thioacetamide (i.e., —SCH₂C(O)NH—), a thioacetamide moiety substituted with an alkyl group (i.e., —SCH(Rᵣ)C(O)NH—, wherein Rᵣ is a unsubstituted C₁-C₂ alkyl) a sulfonyl acetamide, or a thioethan-1-amino moiety,
B8 and B9 are independently unsubstituted aroxy, substituted aroxy, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, or unsubstituted C₃-C₂₀ cycloalkynyl, or fused combinations thereof.

2. The composition of claim 1, wherein for Formula I, R₁, R₂, and R₃, are independently hydrogen, substituted alkyl, unsubstituted alkyl, substituted aryl, unsubstituted aryl, substituted heteroaryl, unsubstituted heteroaryl, substituted polyaryl, unsubstituted polyaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, or fused combinations thereof.

3. The composition of claim 1, wherein for Formula I, R₁, R₂, and R₃, are independently hydrogen, substituted alkyl, unsubstituted alkyl,

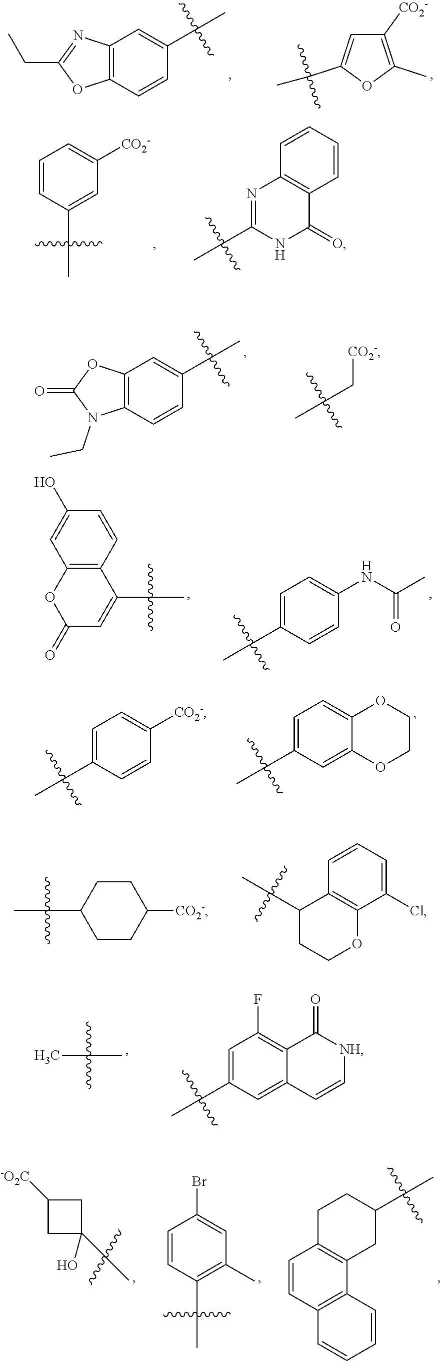

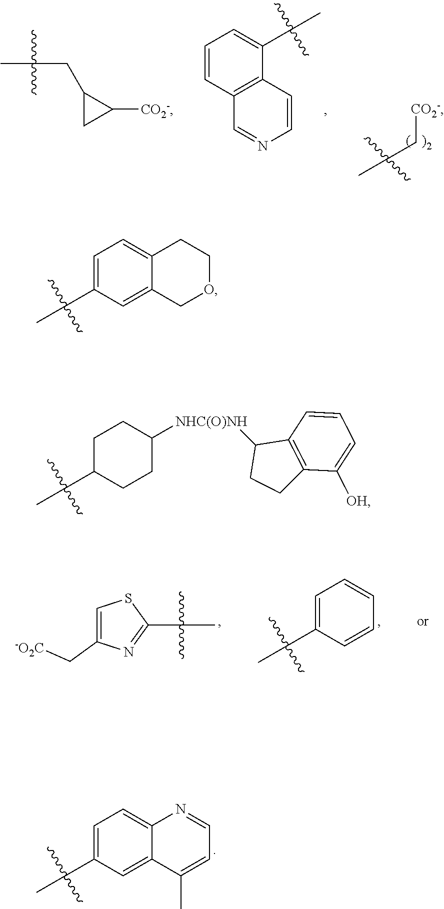

4. The composition of claim 1, wherein for Formula I, A₁ and A₂ are independently absent, substituted alkyl, unsubstituted alkylene, substituted aryl, unsubstituted aryl, substituted heteroaryl, unsubstituted heteroaryl, or —NR_{A1}—, wherein R_{A1} is hydrogen, unsubstituted alkyl, or substituted alkyl.

5. The composition of claim 1, wherein for Formula I, the compound has a structure:

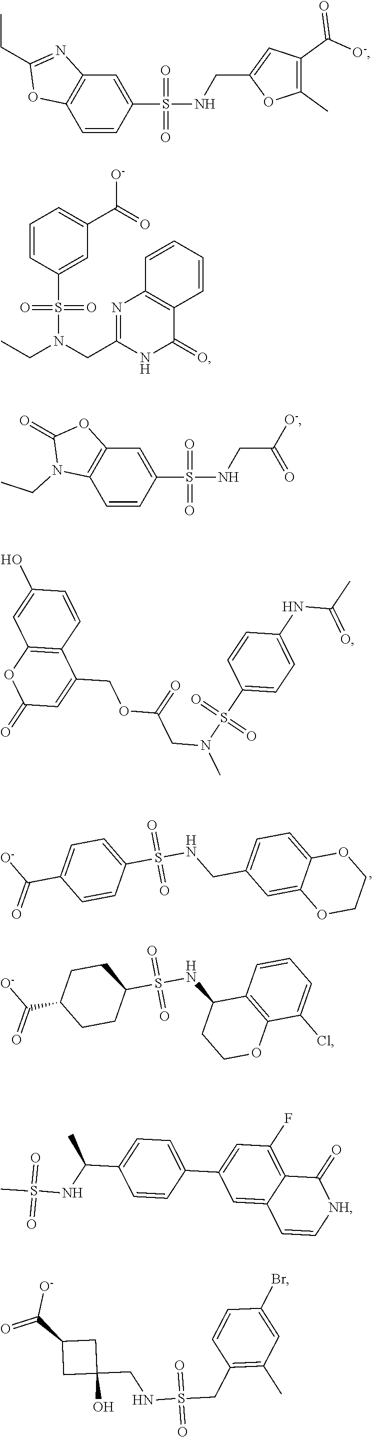

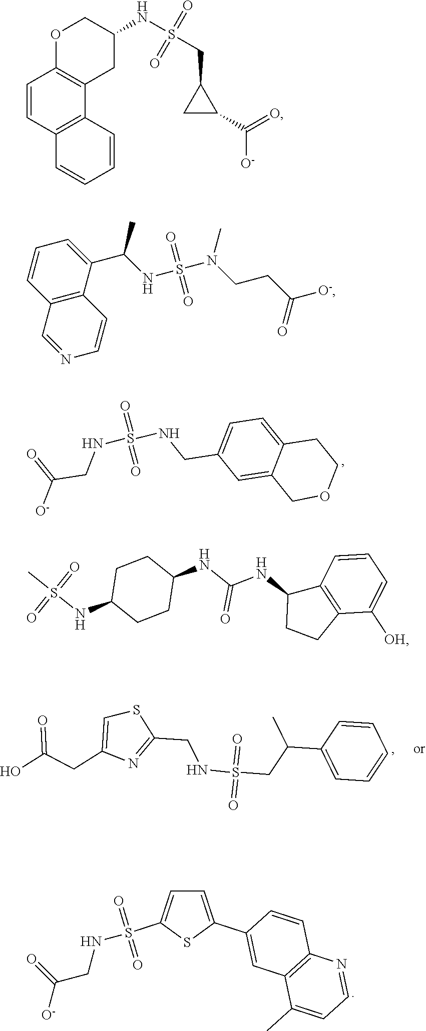

6. The composition of claim 1, wherein for Formula II,
(a) the dashed line denotes the presence of a bond and R₄ is absent,
(b) B1 is substituted aryl, unsubstituted aryl, substituted heteroaryl, unsubstituted heteroaryl, or fused combinations thereof, and/or
(c) R₅ is substituted aryl, unsubstituted aryl, substituted heteroaryl, unsubstituted heteroaryl, or fused combinations thereof.

7. The composition of claim 1, wherein for Formula II,
(a) R₅ has a structure: 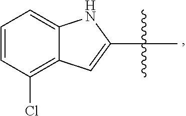 [SMILES: *c1cc2c(Cl)cccc2[nH]1]
and/or
(b) A_{2c} is —NR_{A2c}—, wherein R_{A2c} is hydrogen.

8. The composition of claim 1, wherein for Formula II, the compound has a structure:

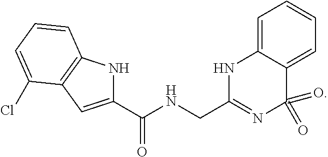

9. The composition of claim 1, wherein for Formula III,
(a) R₆ and R₉ are independently absent, substituted carbonyl,
substituted alkyl, unsubstituted alkylene, substituted alkylthio, substituted aryl, unsubstituted aryl, substituted heteroaryl, or unsubstituted heteroaryl, and/or
(b) R_{6′} and R_{9′} are independently unsubstituted alkyl, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

10. The composition of claim 1, wherein for Formula III, R_{6′} and R_{9′} are independently

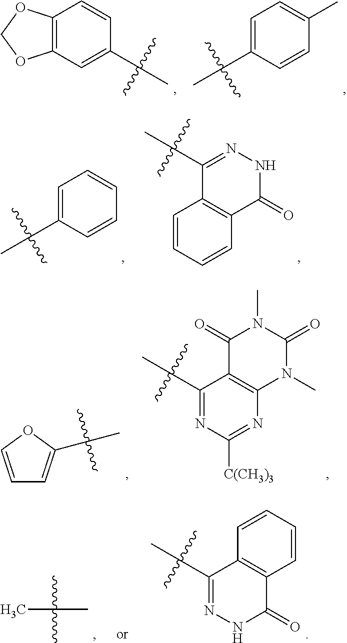

11. The composition of claim 1, wherein for Formula III, the compound has a structure:

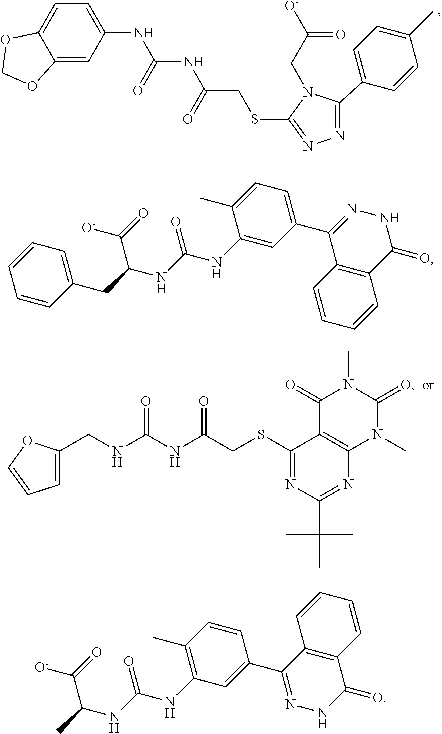

12. The composition of claim 1, wherein for Formula IV,
(a) A₅ and A_{5′} are independently absent, substituted alkyl, unsubstituted alkyl, substituted alkylene, unsubstituted alkylene, or substituted C₁-C₂₀ heterocyclyl,
(b) R_{A5}, R₁₀ and R₁₂ are independently absent, hydrogen, unsubstituted alkyl, or substituted alkyl, and/or
(c) R₁₁ and R₁₃ are independently substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

13. The composition of claim 1, wherein for Formula IV, R₁₁ and R₁₃ are independently

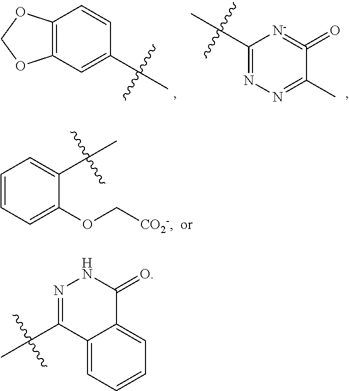

14. The composition of claim 1, wherein for Formula IV, the compound has a structure:

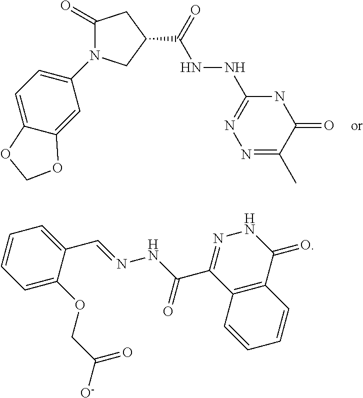 [SMILES: Cc1nnc(NNC(=O)[C@H]2CC(=O)N(c3ccc4c(c3)OCO4)C2)[nH]c1=O ; O=C([O-])COc1ccccc1/C=N/NC(=O)c1n[nH]c(=O)c2ccccc12]

15. The composition of claim 1, wherein for Formula V,
(a) B2 and B3 are independently substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, or fused combinations thereof, and/or
(b) A₆ is a single bond, —NH—, unsubstituted carbonyl, substituted carbonyl, unsubstituted amide, substituted amide, substituted alkyl, unsubstituted alkylthio, substituted alkylthio, substituted aryl, unsubstituted aryl, substituted heteroaryl, unsubstituted heteroaryl, substituted C₃-C₂₀ cycloalkyl, unsubstituted C₃-C₂₀ cycloalkyl, substituted C₁-C₂₀ heterocyclyl, or unsubstituted C₁-C₂₀ heterocyclyl.

16. The composition claim 1, wherein for Formula V, B2 and B3 are independently

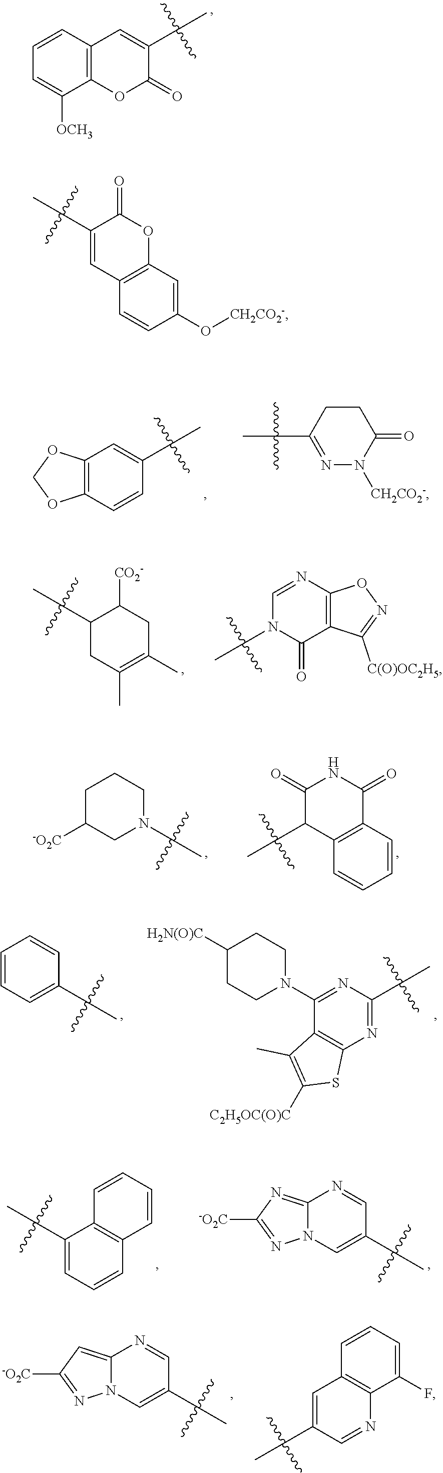

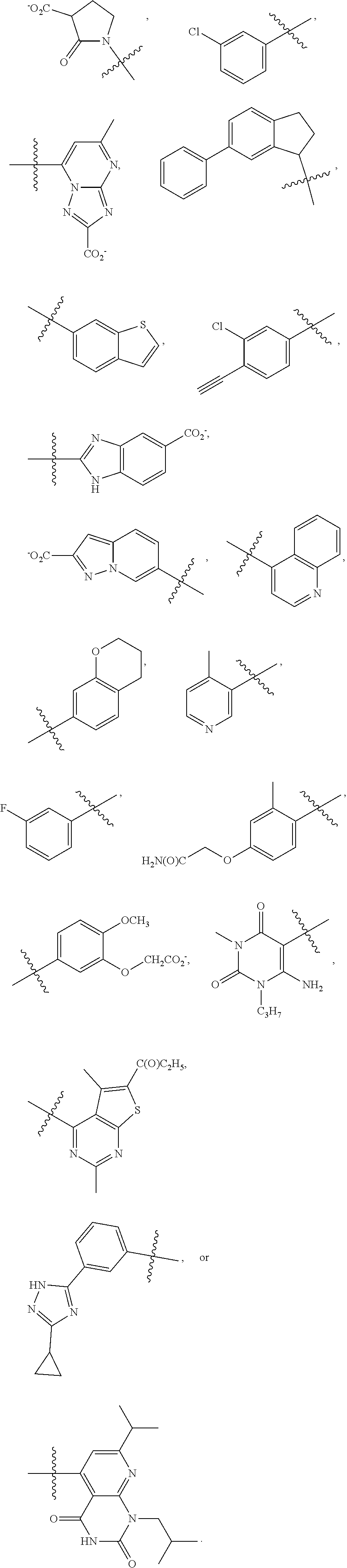

17. The composition of claim 1, wherein for Formula V, the compound has a structure:

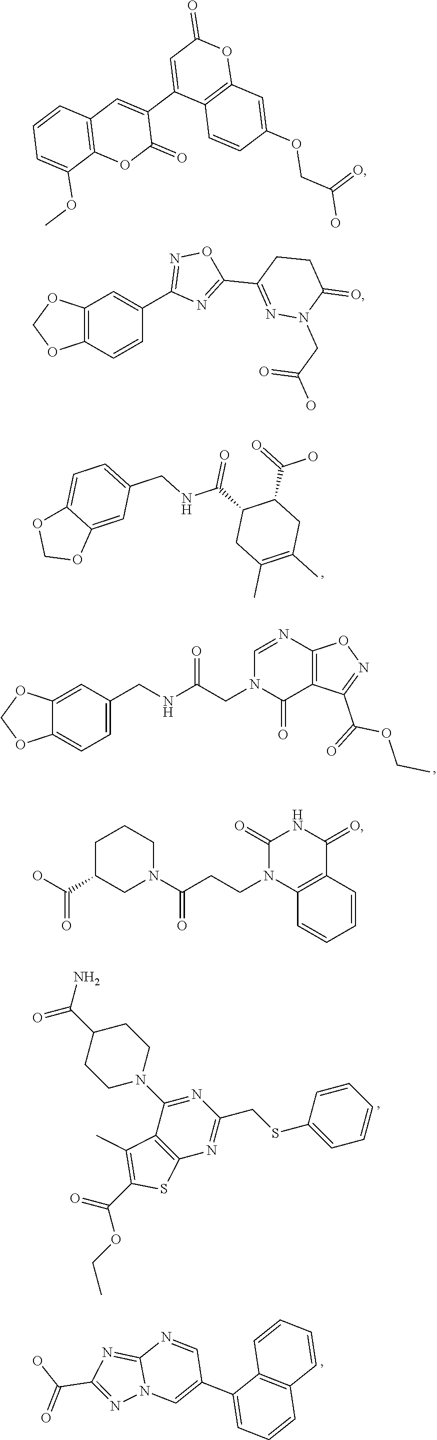

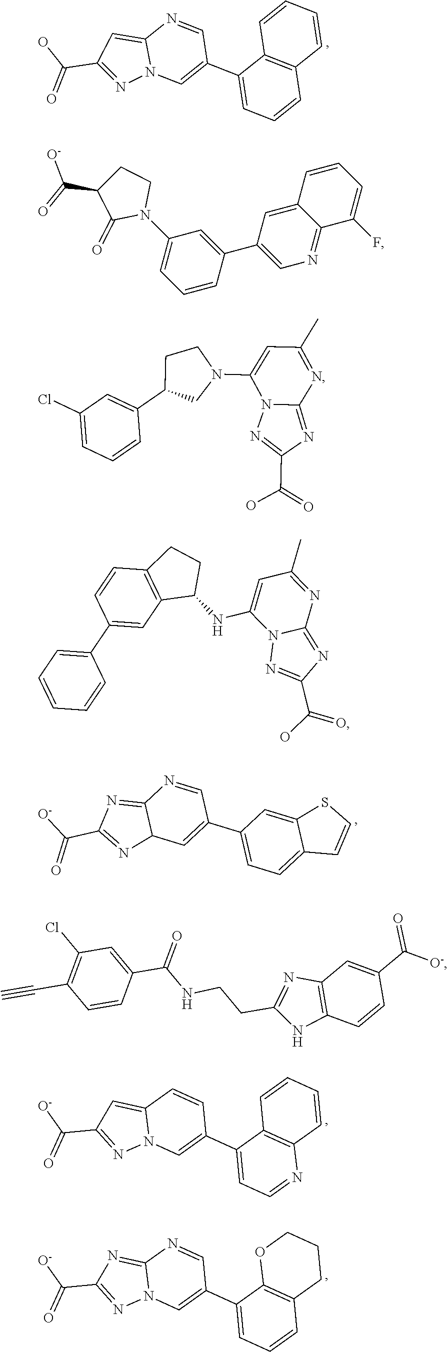

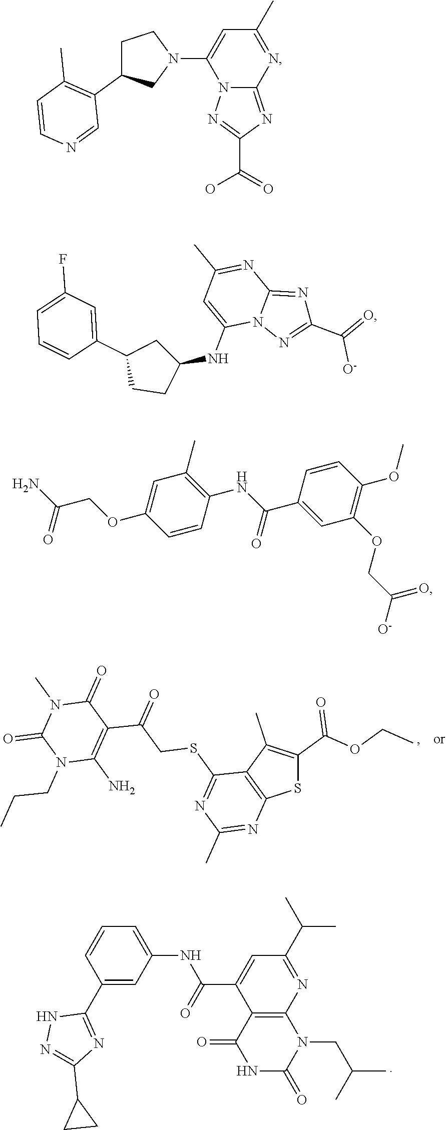 [SMILES: Cc1cc(N2CC[C@H](c3cnccc3C)C2)n2nc(C(=O)O)nc2n1 ; Cc1cc(N[C@H]2CC[C@H](c3cccc(F)c3)C2)n2nc(C(=O)[O-])nc2n1 ; COc1ccc(C(=O)Nc2ccc(OCC(N)=O)cc2C)cc1OCC(=O)[O-] ; CCCn1c(N)c(C(=O)CSc2nc(C)nc3sc(C(=O)OCC)c(C)c23)c(=O)n(C)c1=O ; CC(C)Cn1c(=O)[nH]c(=O)c2c(C(=O)Nc3cccc(-c4nc(C5CC5)n[nH]4)c3)cc(C(C)C)nc21]

18. The composition of claim 1, wherein for Formula VI,
(a) R₁₄ and R₁₅ are organic moieties each bonded to the S(═O)₂ group via a carbon atom, and are independently unsubstituted alkyl, substituted alkyl, unsubstituted aryl, substituted aryl, substituted carbonyl, unsubstituted carbonyl, unsubstituted heteroaryl, substituted heteroaryl, or fused combinations thereof, and/or
(b) R_{14′} and R_{15′} are independently absent, hydrogen, unsubstituted carboxyl, substituted carboxyl, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof, wherein at least one of R_{14′} and R_{15′} is substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

19. The composition of claim 1, wherein for Formula VI, R_{14′} and R_{15′} are independently absent, hydrogen,

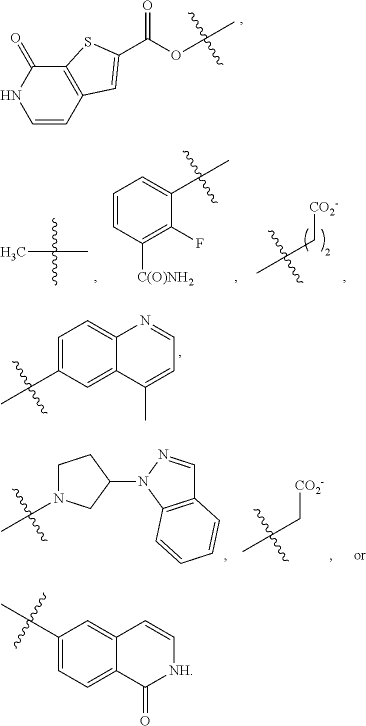

20. The composition of claim 1, wherein for Formula VII,
(a) B4 contains between four and eight carbon atoms, such as a thiaspiro[3.5]nonane and a tetrahydrothiophene,
(b) n is an integer between 2 and 5,
(c) each R₁₆ is independently absent, substituted alkyl, unsubstituted alkyl, substituted amide, or unsubstituted amide, and/or
(d) each R_{16′} is independently unsubstituted carboxyl, substituted carboxyl, unsubstituted alkyl, substituted alkyl, substituted aryl, unsubstituted aryl, substituted heteroaryl, unsubstituted heteroaryl, or fused combinations thereof.

21. The composition of claim 1, wherein for Formula VII, each R_{16′} is independently

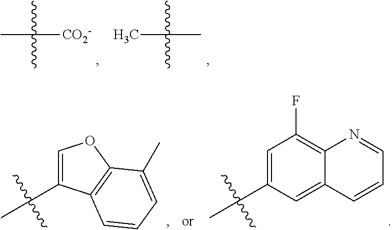

22. The composition of claim 1, wherein for Formula VII, wherein the compound has a structure:

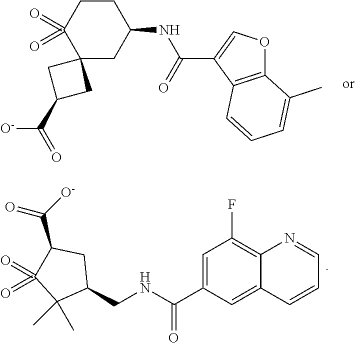

23. The composition of claim 1, wherein for Formula VIII,
(a) the dashed line denotes the absence of a ring system and B6 is not present, and A₇ is unsubstituted alkyl, substituted alkyl, or unsubstituted alkylene, and/or
(b) B5 and B7 are independently substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

24. The composition of claim 1, wherein for Formula VIII, B5 and B7 are independently

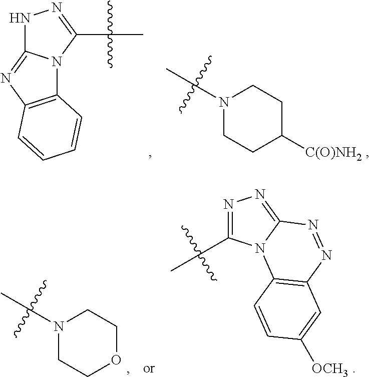

25. The composition of claim 1, wherein for Formula VIII, the compound has a structure:

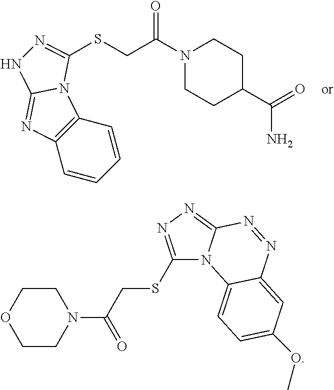 [SMILES: NC(=O)C1CCN(C(=O)CSc2n[nH]c3nc4ccccc4n23)CC1 ; COc1ccc2c(c1)nnc1nnc(SCC(=O)N3CCOCC3)n12]

26. The composition of claim 1, wherein for Formula VIII, the dashed line denotes the presence of a ring system and B6 is present, wherein:
A₇ is a carbon atom bonded to one or no hydrogen atom according to valency,
B6 is substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

27. The composition of claim 1, wherein for Formula VIII,
(a) B6 is substituted heteroaryl, and/or
(b) B5 and B7 are independently substituted heteroaryl, unsubstituted heteroaryl, substituted C₁-C₂₀ heterocyclyl, unsubstituted C₁-C₂₀ heterocyclyl, or fused combinations thereof.

28. The composition of claim 1, wherein for Formula VIII, wherein B5 and B7 are independently

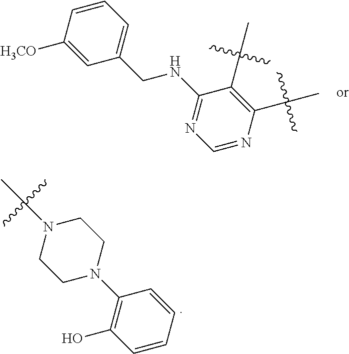 [SMILES: *c1ncnc(NCc2cccc(OC)c2)c1* ; *N1CCN(c2ccccc2O)CC1]

29. The composition of claim 1, wherein for Formula VIII, the compound has a structure:

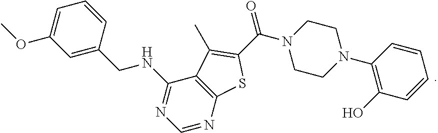 [SMILES: COc1cccc(CNc2ncnc3sc(C(=O)N4CCN(c5ccccc5O)CC4)c(C)c23)c1]

30. The composition of claim 1, wherein for Formula IX, B8 and B9 are independently unsubstituted aroxy, substituted aroxy, substituted aryl, unsubstituted aryl, substituted heteroaryl, unsubstituted heteroaryl, substituted C₁-C₂₀ heterocyclyl, unsubstituted C₁-C₂₀ heterocyclyl, or fused combinations thereof.

31. The composition of claim 1, wherein for Formula IX, B8 and B9 are independently

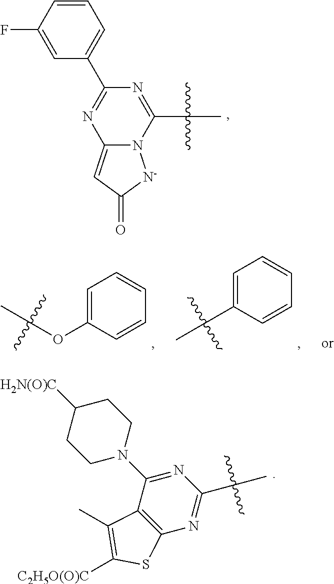

32. The composition of claim 1, wherein for Formula IX, the compound has a structure:

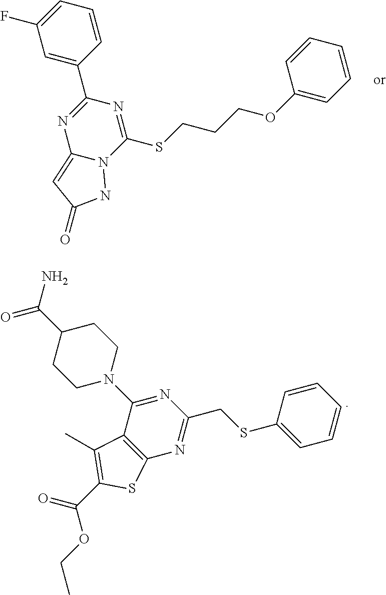

33. The composition of claim 1, wherein:
(a) the compound has a topological polar surface area (i) between 70 Å and 140 Å, or (ii) greater than 140 Å,
(b) the compound has a molecular weight (i) between 200 Da and 500 Da, or (ii) greater than 500 Da and no more than 2,500 Da, and/or
(c) the compound has one or more of hydrogen bond donors, hydrogen bond acceptors, molecular weight, and octanol-water partition coefficient non-conforming with Lipinski's rule of five.

34. A method of modulating ENPP1 activity in a subject in need thereof, the method comprising administering to the subject the composition of claim 1, the composition containing an effective amount of the compound or the pharmaceutically acceptable salt thereof.
